SEQUENCE LISTING (22 sequences)

SEQ ID NO: 1 - protein, 328 aa, Caulobacter crescentus
MDDASSLYDAARFKRAGRGKIYDSIIDTIGDTPLVRLPRLSAELNPKAEVLAKLEFFNPIASVKDRIGVSMIESLEAQGVLKPGATIIEPTSGNTGIALAFVAAAKGYKLTLVMPESMSIERRKMLLLLGAKLELTPAEKGMRGAVTRAQELIEATPGAVMPQQFENSANPLIHRVSTAEEIWNDTAGAVDAVVSGVGTGGTITGVGQALKARKPSLKMVAVEPEASPVLSGGAPGPHKIQGIGAGFVPGVLDRGVIDDIVQVSNDDAFAMARRAAATEGLPVGISSGAALTAAFDLALRDAYKGKTIVVIIPSFAERYLSTALFDGL

SEQ ID NO: 2 - protein, 328 aa, Caulobacter crescentus
MDDASSLYDAARFKRAGRGKIYDSIIDTIGDTPLVRLPRLSAELNPKAEVLAKLEFFNPIASVKDRIGVSMIESLEAQGVLKPGATIIEPTSGNTGIALAVVAAAKGYKLTLVMPESMSIERRKMLLLLGAKLELTPAEKGMRGAVTRAQELIEATPGAVMPQQFENSANPLIHRVSTAEEIWNDTAGAVDAVVSGVGTGGTITGVGQALKARKPSLKMVAVEPEASPVLSGGAPGPHKIQGIGAGFVPGVLDRGVIDDIVQVSNDDAFAMARRAAATEGLPVGISSGAALTAAFDLALRDAYKGKTIVVIIPSFAERYLSTALFDGL

SEQ ID NO: 3 - protein, 328 aa, Caulobacter crescentus
MDDASSLYDAARFKRAGRGKIYDSIIDTIGDAPLVRLPRLSAELNPKAEVLAKLEFFNPIASVKDRIGVSMIESLEAQGVLKPGATIIEPTSGNTGIALAFVAAAKGYKLTLVMPESMSIERRKMLLLLGAKLELTPAEKGMRGAVTRAQELIEATPGAVMPQQFENSANPLIHRVSTAEEIWNDTAGAVDAVVSGVGTGGTITGVGQALKARKPSLKMVAVEPEASPVLSGGAPGPHKIQGIGAGFVPGVLDRGVIDDIVQVSNDDAFAMARRAAATEGLPVGISSGAALTAAFDLALRDAYKGKTIVVIIPSFAERYLSTALFDGL

SEQ ID NO: 4 - protein, 327 aa, Caulobacter crescentus
MDDASSLYDAARFKRAGRGKIYDSIIDTIGDTPLVRLPRLSAELNPKAEVLAKLEFFNPIASVKDRIGVSMIESLEAQGVLKPGATIIEPTSGNTGIALAFVAAAKGYKLTLVMPESMSIERRKMLLLGAKLELTPAEKGMRGAVTRAQELIEATPGAVMPQQFENSANPLIHRVSTAEEIWNDTAGAVDAVVSGVGTGGTITGVGQALKARKPSLKMVAVEPEASPVLSGGAPGPHKIQGIGAGFVPGVLDRGVIDDIVQVSNDDAFAMARRAAATEGLPVGISSGAALTAAFDLALRDAYKGKTIVVIIPSFAERYLSTALFDGL

SEQ ID NO: 5 - protein, 328 aa, Caulobacter crescentus
MDDASSLYDAARFKRAGRGKIYDSIIDTIGDTPLVRLPRLSAELNPKAEVLAKLEFFNPIASVKDRIGVSMIESLEAQGVLKPGATIIEPTSGNTGIALAFVAAAKGYKLTLVMPESMSIERRKMLLLLGAKLELTPAEKGMRGAVTRAQELIEATPGAVMPQQFENSANPLIHRVSTAEEIWNDTAGAVDAVVSGVGTGGTITGVGQALKARKPSLKMVAVEPEASPVLSGGAPGPHKIQGIGAGFVPGVLDRGVIDDIVQVSNDDAFATARRAAATEGLPVGISPGAALTAAFDLALRDAYKGKTIVVIIPSFAERYLSTALFDGL

SEQ ID NO: 6 - protein, 340 aa, Caulobacter crescentus
LQTRLEGANSQTRTSAGAFRKTMERPNERRRLPPLNALRAFEAAARHLNFSRAADELSVTPGAVSQQIQNLEDYVGAALFKRTPKGLLLTDAAQTALPALREAFDRLAEAASLLTAAVDGRRLTLTAAPSFAAKWLVPRLGKFEQAHPQVDVWLSAGMEVVDFATGEVDMAIRYGSGRYPGLEVIRLLHETVVPVASPELLEQNALERPEDLQHHILLHDGSPDADDSCPDWAMWLAARGIRGVDGARGPRFNQSSLVIEAAANGRGIALAKRTLAQADLDSGRLVIPFEASTAVDFAYYVVHPKAKGRLPQVKAFVNWLKAEAEAHEAALRTLDNGAGI

SEQ ID NO: 7 - protein, 340 aa, Caulobacter crescentus
LQTRLEGANSQTRTSAGAFRKTMERPNERRRLPPLNALRAFEAAARHLNFSRAADELSVTPGAVSQQIQNLEDYVGAALFKRTPKGLLLTDAAQTALPALREAFDRLAEAASLLTAAVDGRRLTLTAAPSFAAKWLVPRLGKFEQAHPQVDVWLSAGMEVVDFATGEVDMAIRYGSGRYPGLEVIRLLHETVVPVASPELLEQNALERPEDLQHHILLHDGSPDADDSCPDWAMWLAARGIRGVDGARGPRFNQFSLVIEAAANGRGIALAKRTLAQADLDSGRLVIPFEASTAVDFAYYVVHPKAKGRLPQVKAFVNWLKAEAEAHEAALRTLDNGAGI

SEQ ID NO: 8 - protein, 340 aa, Caulobacter crescentus
LQTRLEGANSQTRTSAGAFRKTMERPNERRRLPPLNALRAFEAAARHLNFSRAADELSVTPGAVSQQIQNLEDYVGAALFKRTPKGLLLTDAAQTALPALREAFDRLAEAASLLTAAVDGRRLTLTAAPSFAAKWLVPRLGKFEQAHPQVDVWLSAGMEVVDFATGEVDMAIRYGSGRYPGLKVIRLLHETVVPVASPELLEQNALERPEDLQHHILLHDGSPDADDSCPDWAMWLAARGIRGVDGARGPRFNQSSLVIEAAANGRGIALAKRTLAQADLDSGRLVIPFEASTAVDFAYYVVHPKAKGRLPQVKAFVNWLKAEAEAHEAALRTLDNGAGI

SEQ ID NO: 9 - protein, 257 aa, Caulobacter crescentus
LQTRLEGANSQTRTSAGAFRKTMERPNERRRLPPLNALRAFEAAARHLNFSRAADELSVTPGAVSQQIQNLEDYVGAALFKRTPKGLLLTDAAQTALPALREAFDRLAEAASLLTAAVDGRRLTLTAAPSFAAKWLVPRLGKFEQAHPQVDVWLSAGMEVVDFATGEVDMAIRYGSGRYPGLEVIRLLHETVVPVASPELLEQNALERPEDLQHHILLHDGSPDADDSCPDWAMWLAARGIRGVDGARGPASTNPAW

SEQ ID NO: 10 - protein, 340 aa, Caulobacter crescentus
LQTRLEGANSQTRTSAGAFRKTMERPNERRRLPPLNALRAFEAAARHLNFSRAADELSVTPGAVSQQIQNLEDYVGAALFKRTPKGLLLTDAAQTALPALREAFDRLAEAASLLTAAVDGRRLTLTAAPSFAAKWLVPRLGKFEQAHPQVDVWLSAGMEVVDFATGEVDMAIRYGSGRYPGLEVIRLLHETVVPVASPELLEQNALERPEDLQHHILLHDGSPDADDSCPDWAMWLAARGIRGVDGARAPRFNQSSLVIEAAANGRGIALAKRTLAQADLDSGRLVIPFEASTAVDFAYYVVHPKAKGRLPQVKAFVNWLKAEAEAHEAALRTLDNGAGI

SEQ ID NO: 11 - protein, 54 aa, Caulobacter crescentus
MAHQSALIAEDIEAYLHQHQHKSLLRFITCGSVDDGKSTLIGRLLYDSKMIFED

SEQ ID NO: 12 - DNA, 987 bp, Caulobacter crescentus
atggacgacgcttcctccctctatgacgccgcccgcttcaagcgcgctggccgtggcaagatctatgactcgatcatcgacacgatcggtgacaccccgctggtgcgcctgccgcgtctgtccgccgagctgaaccccaaggccgaggtcctggccaagctggagttcttcaacccgatcgcctcggtgaaggaccggatcggcgtctcgatgatcgagtcgctggaggctcagggcgtcttgaagccgggcgcgacgattatcgagccgacctcgggcaacaccggcatcgccctggccgtcgtcgcggcggccaagggctacaagctgaccctggtcatgcccgagagcatgtcgatcgagcgccgcaagatgctgctgctgctcggcgccaagctggaactgaccccggccgagaagggcatgcgcggcgcggtcacccgggcccaggagctgatcgaggcgaccccgggcgcggtgatgccgcaacagttcgaaaacagcgccaatccgctgatccatcgcgtctcgaccgccgaggagatctggaacgacaccgccggcgcggtcgacgcggtggtctcgggcgtcgggaccggcggcaccatcacgggcgttggccaggcgctgaaggcgcgcaagccctcgctgaagatggtggcggtggagcccgaggcctcgccggtgctgtcgggcggcgcgccgggtccgcacaagatccaggggatcggcgcgggcttcgtgcccggcgtgctggatcgcggcgtcatcgacgacatcgtccaggtcagcaatgacgacgccttcgccatggcccgccgcgcggcggccaccgaagggttgccggtcggcatcagctccggcgcggccctgacggccgcgttcgatctggccctgcgcgatgcgtacaagggcaagacgatcgtggtgatcatccccagcttcgccgagcgctacctgtcgacggctttgttcgacgggctctag

SEQ ID NO: 13 - DNA, 987 bp, Caulobacter crescentus
atggacgacgcttcctccctctatgacgccgcccgcttcaagcgcgctggccgtggcaagatctatgactcgatcatcgacacgatcggtgacgccccgctggtgcgcctgccgcgtctgtccgccgagctgaaccccaaggccgaggtcctggccaagctggagttcttcaacccgatcgcctcggtgaaggaccggatcggcgtctcgatgatcgagtcgctggaggctcagggcgtcttgaagccgggcgcgacgattatcgagccgacctcgggcaacaccggcatcgccctggccttcgtcgcggcggccaagggctacaagctgaccctggtcatgcccgagagcatgtcgatcgagcgccgcaagatgctgctgctgctcggcgccaagctggaactgaccccggccgagaagggcatgcgcggcgcggtcacccgggcccaggagctgatcgaggcgaccccgggcgcggtgatgccgcaacagttcgaaaacagcgccaatccgctgatccatcgcgtctcgaccgccgaggagatctggaacgacaccgccggcgcggtcgacgcggtggtctcgggcgtcgggaccggcggcaccatcacgggcgttggccaggcgctgaaggcgcgcaagccctcgctgaagatggtggcggtggagcccgaggcctcgccggtgctgtcgggcggcgcgccgggtccgcacaagatccaggggatcggcgcgggcttcgtgcccggcgtgctggatcgcggcgtcatcgacgacatcgtccaggtcagcaatgacgacgccttcgccatggcccgccgcgcggcggccaccgaagggttgccggtcggcatcagctccggcgcggccctgacggccgcgttcgatctggccctgcgcgatgcgtacaagggcaagacgatcgtggtgatcatccccagcttcgccgagcgctacctgtcgacggctttgttcgacgggctctag

SEQ ID NO: 14 - DNA, 984 bp, Caulobacter crescentus
atggacgacgcttcctccctctatgacgccgcccgcttcaagcgcgctggccgtggcaagatctatgactcgatcatcgacacgatcggtgacaccccgctggtgcgcctgccgcgtctgtccgccgagctgaaccccaaggccgaggtcctggccaagctggagttcttcaacccgatcgcctcggtgaaggaccggatcggcgtctcgatgatcgagtcgctggaggctcagggcgtcttgaagccgggcgcgacgattatcgagccgacctcgggcaacaccggcatcgccctggccttcgtcgcggcggccaagggctacaagctgaccctggtcatgcccgagagcatgtcgatcgagcgccgcaagatgctgctgctcggcgccaagctggaactgaccccggccgagaagggcatgcgcggcgcggtcacccgggcccaggagctgatcgaggcgaccccgggcgcggtgatgccgcaacagttcgaaaacagcgccaatccgctgatccatcgcgtctcgaccgccgaggagatctggaacgacaccgccggcgcggtcgacgcggtggtctcgggcgtcgggaccggcggcaccatcacgggcgttggccaggcgctgaaggcgcgcaagccctcgctgaagatggtggcggtggagcccgaggcctcgccggtgctgtcgggcggcgcgccgggtccgcacaagatccaggggatcggcgcgggcttcgtgcccggcgtgctggatcgcggcgtcatcgacgacatcgtccaggtcagcaatgacgacgccttcgccatggcccgccgcgcggcggccaccgaagggttgccggtcggcatcagctccggcgcggccctgacggccgcgttcgatctggccctgcgcgatgcgtacaagggcaagacgatcgtggtgatcatccccagcttcgccgagcgctacctgtcgacggctttgttcgacgggctctag

SEQ ID NO: 15 - DNA, 987 bp, Caulobacter crescentus
atggacgacgcttcctccctctatgacgccgcccgcttcaagcgcgctggccgtggcaagatctatgactcgatcatcgacacgatcggtgacaccccgctggtgcgcctgccgcgtctgtccgccgagctgaaccccaaggccgaggtcctggccaagctggagttcttcaacccgatcgcctcggtgaaggaccggatcggcgtctcgatgatcgagtcgctggaggctcagggcgtcttgaagccgggcgcgacgattatcgagccgacctcgggcaacaccggcatcgccctggccttcgtcgcggcggccaagggctacaagctgaccctggtcatgcccgagagcatgtcgatcgagcgccgcaagatgctgctgctgctcggcgccaagctggaactgaccccggccgagaagggcatgcgcggcgcggtcacccgggcccaggagctgatcgaggcgaccccgggcgcggtgatgccgcaacagttcgaaaacagcgccaatccgctgatccatcgcgtctcgaccgccgaggagatctggaacgacaccgccggcgcggtcgacgcggtggtctcgggcgtcgggaccggcggcaccatcacgggcgttggccaggcgctgaaggcgcgcaagccctcgctgaagatggtggcggtggagcccgaggcctcgccggtgctgtcgggcggcgcgccgggtccgcacaagatccaggggatcggcgcgggcttcgtgcccggcgtgctggatcgcggcgtcatcgacgacatcgtccaggtcagcaatgacgacgccttcgccacggcccgccgcgcggcggccaccgaagggttgccggtcggcatcagccccggcgcggccctgacggccgcgttcgatctggccctgcgcgatgcgtacaagggcaagacgatcgtggtgatcatccccagcttcgccgagcgctacctgtcgacggctttgttcgacgggctctag

SEQ ID NO: 16 - DNA, 1023 bp, Caulobacter crescentus
ttgcaaactcggctcgaaggggccaattcgcaaacaaggacttctgccggggcgttcaggaaaactatggaaagaccgaacgagcgccgtcgcctgccgccgctgaacgcccttcgcgcgttcgaggccgccgcgcgccacttgaacttcagccgggccgcagacgagctgtccgtgacccctggcgcggtcagccaacagatccagaacctggaagactatgtcggcgcggcgctgttcaagcgcacgcccaagggcctgctgctgaccgacgcggcgcagaccgccctgcccgccctgcgcgaggcgttcgatcgcctggccgaggccgccagcctgctgaccgccgccgtcgacggccgtcgcctgaccctgaccgccgccccctcgttcgcggccaagtggctggtgccgcgcctgggcaagttcgaacaggcccacccgcaggtcgatgtctggctgtcggcgggcatggaggtcgtagacttcgccaccggcgaggtggacatggccatccgctacggctcaggtcgctatccgggccttgaggtcatccggctgctgcatgagaccgtcgtcccggtcgccagtcccgagctgctcgagcagaacgccctggagcgccccgaggacctgcagcaccacatcctgctgcatgacgggtcacccgacgccgacgacagctgccctgactgggcgatgtggctggcggcgcgcgggatcaggggcgtcgatggcgcgaggggcccccgcttcaaccaatccagcctggtgatcgaggccgccgccaacggccggggcatcgccctggccaagcgcaccctggcccaggccgatctcgactccggacggctggtgatcccgttcgaggcctcgaccgccgtcgacttcgcctactatgtggtccaccccaaggcgaaggggcgactgccgcaagtgaaggcctttgtgaactggctgaaggccgaggccgaggcgcatgaggccgcgctgcgcacgctggacaacggcgcggggatctga

SEQ ID NO: 17 - DNA, 1023 bp, Caulobacter crescentus
ttgcaaactcggctcgaaggggccaattcgcaaacaaggacttctgccggggcgttcaggaaaactatggaaagaccgaacgagcgccgtcgcctgccgccgctgaacgcccttcgcgcgttcgaggccgccgcgcgccacttgaacttcagccgggccgcagacgagctgtccgtgacccctggcgcggtcagccaacagatccagaacctggaagactatgtcggcgcggcgctgttcaagcgcacgcccaagggcctgctgctgaccgacgcggcgcagaccgccctgcccgccctgcgcgaggcgttcgatcgcctggccgaggccgccagcctgctgaccgccgccgtcgacggccgtcgcctgaccctgaccgccgccccctcgttcgcggccaagtggctggtgccgcgcctgggcaagttcgaacaggcccacccgcaggtcgatgtctggctgtcggcgggcatggaggtcgtagacttcgccaccggcgaggtggacatggccatccgctacggctcaggtcgctatccgggccttgaggtcatccggctgctgcatgagaccgtcgtcccggtcgccagtcccgagctgctcgagcagaacgccctggagcgccccgaggacctgcagcaccacatcctgctgcatgacgggtcacccgacgccgacgacagctgccctgactgggcgatgtggctggcggcgcgcgggatcaggggcgtcgatggcgcgaggggcccccgcttcaaccaattcagcctggtgatcgaggccgccgccaacggccggggcatcgccctggccaagcgcaccctggcccaggccgatctcgactccggacggctggtgatcccgttcgaggcctcgaccgccgtcgacttcgcctactatgtggtccaccccaaggcgaaggggcgactgccgcaagtgaaggcctttgtgaactggctgaaggccgaggccgaggcgcatgaggccgcgctgcgcacgctggacaacggcgcggggatctga

SEQ ID NO: 18 - DNA, 1023 bp, Caulobacter crescentus
ttgcaaactcggctcgaaggggccaattcgcaaacaaggacttctgccggggcgttcaggaaaactatggaaagaccgaacgagcgccgtcgcctgccgccgctgaacgcccttcgcgcgttcgaggccgccgcgcgccacttgaacttcagccgggccgcagacgagctgtccgtgacccctggcgcggtcagccaacagatccagaacctggaagactatgtcggcgcggcgctgttcaagcgcacgcccaagggcctgctgctgaccgacgcggcgcagaccgccctgcccgccctgcgcgaggcgttcgatcgcctggccgaggccgccagcctgctgaccgccgccgtcgacggccgtcgcctgaccctgaccgccgccccctcgttcgcggccaagtggctggtgccgcgcctgggcaagttcgaacaggcccacccgcaggtcgatgtctggctgtcggcgggcatggaggtcgtagacttcgccaccggcgaggtggacatggccatccgctacggctcaggtcgctatccgggcctaaaggtcatccggctgctgcatgagaccgtcgtcccggtcgccagtcccgagctgctcgagcagaacgccctggagcgccccgaggacctgcagcaccacatcctgctgcatgacgggtcacccgacgccgacgacagctgccctgactgggcgatgtggctggcggcgcgcgggatcaggggcgtcgatggcgcgaggggcccccgcttcaaccaatccagcctggtgatcgaggccgccgccaacggccggggcatcgccctggccaagcgcaccctggcccaggccgatctcgactccggacggctggtgatcccgttcgaggcctcgaccgccgtcgacttcgcctactatgtggtccaccccaaggcgaaggggcgactgccgcaagtgaaggcctttgtgaactggctgaaggccgaggccgaggcgcatgaggccgcgctgcgcacgctggacaacggcgcggggatctga

SEQ ID NO: 19 - DNA, 1022 bp, Caulobacter crescentus
ttgcaaactcggctcgaaggggccaattcgcaaacaaggacttctgccggggcgttcaggaaaactatggaaagaccgaacgagcgccgtcgcctgccgccgctgaacgcccttcgcgcgttcgaggccgccgcgcgccacttgaacttcagccgggccgcagacgagctgtccgtgacccctggcgcggtcagccaacagatccagaacctggaagactatgtcggcgcggcgctgttcaagcgcacgcccaagggcctgctgctgaccgacgcggcgcagaccgccctgcccgccctgcgcgaggcgttcgatcgcctggccgaggccgccagcctgctgaccgccgccgtcgacggccgtcgcctgaccctgaccgccgccccctcgttcgcggccaagtggctggtgccgcgcctgggcaagttcgaacaggcccacccgcaggtcgatgtctggctgtcggcgggcatggaggtcgtagacttcgccaccggcgaggtggacatggccatccgctacggctcaggtcgctatccgggccttgaggtcatccggctgctgcatgagaccgtcgtcccggtcgccagtcccgagctgctcgagcagaacgccctggagcgccccgaggacctgcagcaccacatcctgctgcatgacgggtcacccgacgccgacgacagctgccctgactgggcgatgtggctggcggcgcgcgggatcaggggcgtcgatggcgcgaggggccccgcttcaaccaatccagcctggtgatcgaggccgccgccaacggccggggcatcgccctggccaagcgcaccctggcccaggccgatctcgactccggacggctggtgatcccgttcgaggcctcgaccgccgtcgacttcgcctactatgtggtccaccccaaggcgaaggggcgactgccgcaagtgaaggcctttgtgaactggctgaaggccgaggccgaggcgcatgaggccgcgctgcgcacgctggacaacggcgcggggatctga

SEQ ID NO: 20 - DNA, 1023 bp, Caulobacter crescentus
ttgcaaactcggctcgaaggggccaattcgcaaacaaggacttctgccggggcgttcaggaaaactatggaaagaccgaacgagcgccgtcgcctgccgccgctgaacgcccttcgcgcgttcgaggccgccgcgcgccacttgaacttcagccgggccgcagacgagctgtccgtgacccctggcgcggtcagccaacagatccagaacctggaagactatgtcggcgcggcgctgttcaagcgcacgcccaagggcctgctgctgaccgacgcggcgcagaccgccctgcccgccctgcgcgaggcgttcgatcgcctggccgaggccgccagcctgctgaccgccgccgtcgacggccgtcgcctgaccctgaccgccgccccctcgttcgcggccaagtggctggtgccgcgcctgggcaagttcgaacaggcccacccgcaggtcgatgtctggctgtcggcgggcatggaggtcgtagacttcgccaccggcgaggtggacatggccatccgctacggctcaggtcgctatccgggccttgaggtcatccggctgctgcatgagaccgtcgtcccggtcgccagtcccgagctgctcgagcagaacgccctggagcgccccgaggacctgcagcaccacatcctgctgcatgacgggtcacccgacgccgacgacagctgccctgactgggcgatgtggctggcggcgcgcgggatcaggggcgtcgatggcgcgagggccccccgcttcaaccaatccagcctggtgatcgaggccgccgccaacggccggggcatcgccctggccaagcgcaccctggcccaggccgatctcgactccggacggctggtgatcccgttcgaggcctcgaccgccgtcgacttcgcctactatgtggtccaccccaaggcgaaggggcgactgccgcaagtgaaggcctttgtgaactggctgaaggccgaggccgaggcgcatgaggccgcgctgcgcacgctggacaacggcgcggggatctga

SEQ ID NO: 21 - DNA, 1908 bp, Caulobacter crescentus
atggcccaccagtccgccctgatcgccgaggatatcgaagcctacctgcatcagcaccagcacaagtcgctgctgcgcttcatcacctgcggcagcgtcgacgacggcaagtccaccctgatcggtcgcctgctgtacgacagcaagatgatcttcgaggactagatggcggccctcgaggtcgactccaggaaggtcggcacccaaggcggcgcgatcgacttcgccttgctggtcgacggtctggccgctgagcgcgagcaaggcatcaccatcgatgtggcctaccgcttcttctcgaccgagaagcgcaagttcatcgtcgccgacacgcccggccacgagcagtacacccgcaacatggtcaccggggcctcgaccgccgacgcggccgtgatcctgatcgatgcgcgcaagggcgtgctgacccagacccgtcgccacagctatctcgtcagcctgctgggcatccgccacgtggtgctggcggtgaacaagatggacctggtcggctgggacaagggcgtgttcgacgccatcgtcgccgactaccgcgccttcgccgaacagatcggcctttcggtgttcaccccgatcccgatctcaggcctcggcggcgacaacatggcgacccgcagcgacgccacgccctggtttgaaggcccgatcctgatggactggctggaagccgtcgaggtcgaggacgaccttcaggccaagcccttccgcatgccggtccagtgggtcaaccgacccaatctcgacttccggggcttctcgggcctgatcgcctcgggttcgatcaagccgggcgaccgtatccgcgccctgccctcgggccgcgaaagccgcgtggcccgcatcgtcaccctgccgggcgatctcgaccaggccgtcgccggccagtcggtcaccctgacgctggaagacgagatcgacatctcgcgcggcgacgtcatcgccgccgctgacgcccccgccccggtggccaaccagttcgaggccaccctcgtctggatggacgacgagccgctgccgccgggccgcacctatctcctgaagctcggcgcgcgcacggtcggcgccagcatcaccgacatcaagcaccgcgtgaacgtcaacacgctggagaagacggccgccaagcgcctcgaactgaacgagatcggggtctgcaatctgtcgctcgaccaagccatcccgttcgaggcctatgccgaaaaccgtcacatgggcgggttcatcctgatcgaccggctgagcaaccgcacggtcggcgcaggtctgatcaacttcgccctgcggcgagccgacaacatccactggcagcacacagatgtgaccaaggtctcgcgggcggcgctgaaacgccagcgcggtcaggtcgtctggctgacgggcctgtcgggcgcggggaagtcgaccatcgccaatctcgtcgaaaagcgcctgcacgccctgggtcgccacacctatctgctggacggcgacaatgtccggcacgggctgaacaaggacctcgggttcaccgaggaggaccgcgtcgagaacatccgccgggtggccgaggtcgccaagctgatggtcgatgcgggcctgatcgtgctgacggccttcatctcgcccttccgggccgagcgccagctggcgcgcgacctgctggagcccggcgagttcatcgaggttttcgtcgacacgccgctggccgtcgccgaggcccgcgacgtcaaggggctctacaagaaggcgcgctcgggccagctcaagaacttcaccggcgtcgatagtccctatgaggcgccggagtcgccagagctgcgcatcgacaccaccgcgatcgatcctgtcgaggcggccgaacggatcgtcgcctggctggagggccaggagatcgactacaccatctaa

SEQ ID NO: 22 - DNA, 987 bp, Caulobacter crescentus
atggacgacgcttcctccctctatgacgccgcccgcttcaagcgcgctggccgtggcaagatctatgactcgatcatcgacacgatcggtgacaccccgctggtgcgcctgccgcgtctgtccgccgagctgaaccccaaggccgaggtcctggccaagctggagttcttcaacccgatcgcctcggtgaaggaccggatcggcgtctcgatgatcgagtcgctggaggctcagggcgtcttgaagccgggcgcgacgattatcgagccgacctcgggcaacaccggcatcgccctggccttcgtcgcggcggccaagggctacaagctgaccctggtcatgcccgagagcatgtcgatcgagcgccgcaagatgctgctgctgctcggcgccaagctggaactgaccccggccgagaagggcatgcgcggcgcggtcacccgggcccaggagctgatcgaggcgaccccgggcgcggtgatgccgcaacagttcgaaaacagcgccaatccgctgatccatcgcgtctcgaccgccgaggagatctggaacgacaccgccggcgcggtcgacgcggtggtctcgggcgtcgggaccggcggcaccatcacgggcgttggccaggcgctgaaggcgcgcaagccctcgctgaagatggtggcggtggagcccgaggcctcgccggtgctgtcgggcggcgcgccgggtccgcacaagatccaggggatcggcgcgggcttcgtgcccggcgtgctggatcgcggcgtcatcgacgacatcgtccaggtcagcaatgacgacgccttcgccatggcccgccgcgcggcggccaccgaagggttgccggtcggcatcagctccggcgcggccctgacggccgcgttcgatctggccctgcgcgatgcgtacaagggcaagacgatcgtggtgatcatccccagcttcgccgagcgctacctgtcgacggctttgttcgacgggctctag

(19) United States
(12) Patent Application Publication
(10) Pub. No.: US 2017/0088827 A1
(43) Pub. Date: Mar. 30, 2017

(54) BACTERIAL STRAIN FOR LEAD PRECIPITATION

(71) Applicant: Rowan University, Glassboro, NJ (US)

(72) Inventors: Gregory Brian Hecht, Glassboro, NJ (US); Mark J. Hickman, Glassboro, NJ (US); Leanna Panepinto, Glassboro, NJ (US); John T. Gregg, Glassboro, NJ (US)

(73) Assignee: Rowan University, Glassboro, NJ (US)

(21) Appl. No.: 15/275,992

(22) Filed: Sep. 26, 2016

Related U.S. Application Data
(60) Provisional application No. 62/232,376, filed on Sep. 24, 2015.

(51) Int. Cl. C12N 9/10; C12N 1/20; C02F 3/34; C22B 3/00; C22B 3/18; C12N 15/74; C12P 3/00

(52) U.S. Cl. CPC C12N 9/1085; C12N 15/74; C12N 1/20; C12Y 205/01047; C12P 3/00; C22B 13/04; C22B 3/18; C02F 3/342; C02F 2101/20

(57) ABSTRACT

The present invention relates to a mutant CC3625 cysteine synthase. Bacteria containing such mutant cysteine synthase can be used for the precipitation of soluble lead.

### RELATED PATENT DOCUMENTS

This patent document claims benefit under 35 U.S.C. §119 (e) to U.S. Provisional Patent Application Ser. No. 62/232,376 filed on Sep. 24, 2015 incorporated herein in its entirety.

### FIELD OF THE INVENTION

The invention relates to biosorption of metal contaminants with immobilized bacteria. In particular, mutant Caulobacter crescentus is used as an efficient lead precipitator.

### BACKGROUND

Heavy metals such as cadmium, lead, copper and zinc draw the attention of the hygienists because of their toxicity. The public health is directly impacted by the occurrence of heavy metals in water and soil even at low concentrations owing to their accumulation in vegetables through soil solution or contact with the contaminated soil, or with water that has leached the contaminated soil.

It has been reported that some microorganisms can immobilize metals up to high concentrations in their cellular materials especially when they are attached on a support. Various culture devices are known to promote the growth of microorganisms on a surface. For example, biological fluidized beds have been used as for the excellent adhesion potentialities for bacteria. The biological fluidized bed is composed of a cylinder packed with inert particles such as sand, anthracite, glass beads, plastic, stone gravels which provide support for microbial growth. However, one of the drawbacks of these existing culture devices is the requirement of nutritive medium for the microorganisms and the resulting release of large amount of microorganisms which may lead to undesirable side effects to the environment and to the public health. Further, the cost of conventional biosorption approaches is often a factor limiting its wide application in the disposal of heavy metals.

Thus, a need exists for a high efficiency, low cost and environmentally friendly approach in the removal of metal contaminants such as lead.

### SUMMARY OF THE INVENTION

The present invention provides for microorganism containing mediums and methods of use thereof to reduce metal contaminants. In one embodiment, the metal contaminants can include lead, mercury, zinc, copper, cadmium, manganese, chromium, cobalt, nickel, silver and even arsenic. In a more preferred embodiment, the metal contaminant is lead.

In another aspect of the invention, the microorganism used in the described methods is non-pathogenic bacteria. In one embodiment, the bacteria is a mutant oligotrophic fresh water such as Staphylococcus, Lactobacillus, Escherichia coli, Bifidobacteria, Bacteroides, Brevibacterium, and Caulobacter. In a preferred embodiment, Caulobacter crescentus mutants are described which can be used for lead precipitation.

An aspect of the invention provides a mutant CC3625 cysteine synthase protein having an amino acid sequence that differs from SEQ ID NO: 1 in a way selected from the group consisting of
a) substitution of alanine in place of threonine at position 32,
b) substitution of valine in place of phenylalanine at position 101,
c) deletion of lysine at position 129, and
d) both substitution of threonine in place of methionine at position 271 and substitution of proline in place of serine at position 287.

An aspect of the invention provides a mutant CC3625 cysteine synthase protein having an amino acid sequence of one or more of SEQ ID NOs: 2, 3, 4, and 5.

An aspect of the invention provides a bacterium having a mutant CC3625 genotype having a nucleotide sequence of one or more of SEQ ID Nos: 12, 13, 14, and 15.

Another aspect of the invention provides a lead hyper-precipitating strain of Caulobacter crescentus that expresses the mutant CC3625 cysteine synthase described herein.

An aspect of the invention provides a lead precipitating mutant CC1117 cysteine synthase protein having an amino acid sequence of one or more of SEQ ID NOs: 7, 8, 9, and 10.

An aspect of the invention provides a bacterium having a mutant CC1117 genotypes having a nucleotide sequence of one or more of SEQ ID Nos: 17, 18, 19, and 20.

Another aspect provides a reactor for reducing the amount of soluble metal contaminant in an aqueous fluid, the reactor comprising a fluid conduit capable of contacting the fluid with the metal hyper-precipitating strain of the present invention. In one embodiment, a reactor is provided for reducing the amount of soluble lead in an aqueous fluid, the reactor comprising a fluid conduit capable of contacting the fluid with the lead hyper-precipitating strain of the present invention.

Another aspect of the invention provides a method of generating a lead hyper-precipitating strain of Caulobacter crescentus, the method comprising exposing bacteria of a first strain of Caulobacter crescentus to a mutagen, thereafter selecting exposed bacteria which exhibit greater cysteine synthase activity than do bacteria of the first strain not exposed, and culturing the selected bacteria to induce their proliferation, whereby the proliferated bacteria are the lead hyper-precipitating strain.

Another aspect of the invention provides a method of generating a lead hyper-precipitating strain of Caulobacter crescentus, the method comprising exposing bacteria of a first strain of Caulobacter crescentus to a mutagen culturing exposed bacteria in the presence of soluble lead, and thereafter selecting cultured bacteria which exhibit greater lead precipitation than do bacteria of the first strain not exposed, whereby the selected bacteria are the lead hyper-precipitating strain.

Another aspect of the invention provides a method of reducing the amount of soluble lead in an aqueous fluid, the method comprising contacting the fluid with the lead hyper-precipitating strain of Caulobacter crescentus described herein and thereafter separating the liquid and the strain.

Another aspect of the invention provides a method of synthesizing Pb₉(PO₄)₆ (hereafter referred to as lead hexaphosphate), comprising contacting a lead-containing fluid with the lead hyper-precipitating strain of Caulobacter crescentus described herein and isolating the precipitated lead hexaphosphate.

Another aspect of the invention provides a method of inducing wild-type strains of Caulobacter crescentus in reducing the amount of soluble lead in an aqueous fluid, the method comprising exposing bacteria of a first strain of Caulobacter crescentus to an effective amounts of cysteine in a culture medium and culturing the exposed bacteria in the presence of soluble lead, and thereafter selecting cultured bacteria which exhibit greater lead precipitation than do bacteria of the first strain not exposed, whereby the selected bacteria are the lead hyper-precipitating strain.

### BRIEF DESCRIPTION OF THE DRAWINGS

FIG. 1 illustrates the lead precipitation effect of a mutant strain RCCR3 (labeled B) in comparison with a wild-type strain CB 15 (labeled A), and the non-precipitator RCCR263 (labeled C).

FIG. 2 illustrates the transductional mapping of the C. crescentus genome. A locus has been identified which is linked with the brown Hyp (hyper-precipitating) phenotype. A separate locus linked with the white Nop (non-precipitating) phenotype has also been identified.

FIG. 3 illustrates each of the brown hyp mutants for CC3625. This suggests that this gene is necessary and sufficient for the brown lead precipitating phenotype.

FIG. 4 illustrates five of the white hyp nop strains having a mutation in CC1117.

FIG. 5 illustrates the sequence alignment of cysteine synthase proteins (full length) with the arrows show the position of the hyp mutations in RCCR3, RCCR4 and RCCR5.

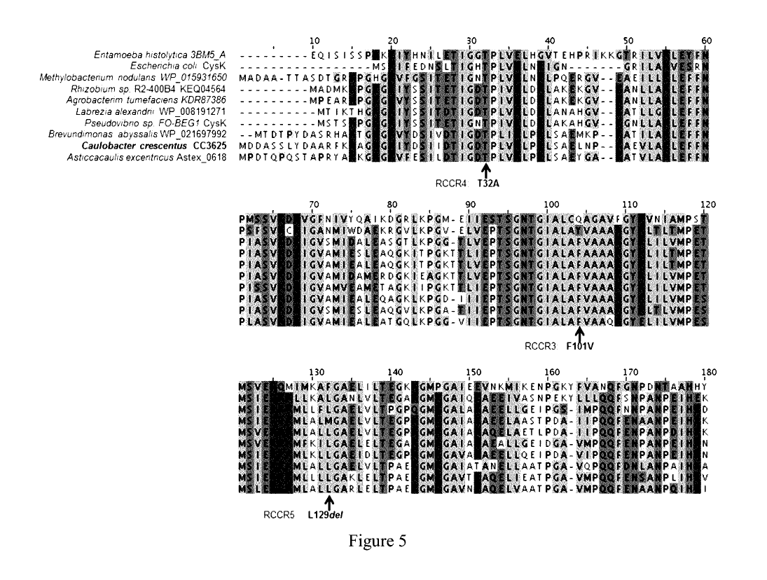

FIG. 6 illustrates the sequence alignment of cysteine synthase proteins (full length) with the arrows show the position of the hyp mutations in RCCR7.

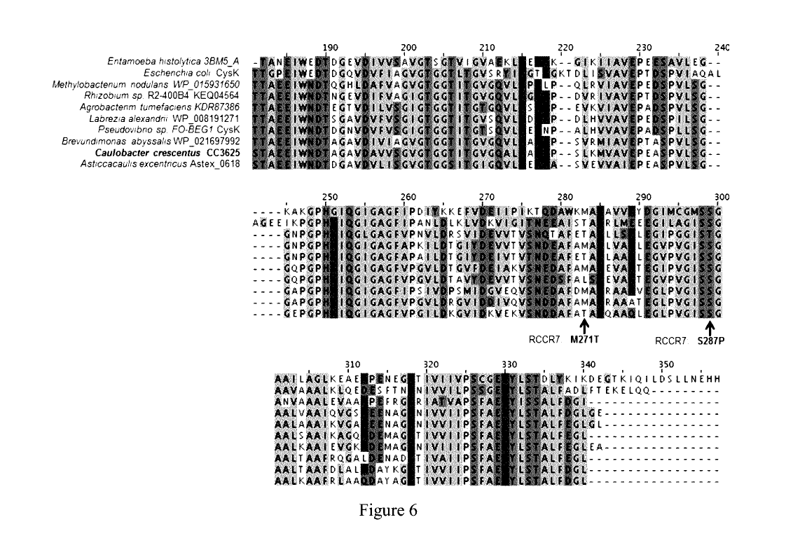

FIG. 7 illustrates the lead precipitation effect of a wild-type CB 15 (labeled A), the hyper-precipitator RCCR3 (labeled B), the non-precipitator RCCR263 (labeled C), NA1000 (labeled D), and the NA1000 ΔphoY deletion strain YJ10010 (labeled E).

### DETAILED DESCRIPTION OF PREFERRED EMBODIMENTS

Various embodiments of the invention provide microorganisms and methods of use thereof for removal of heavy metal contaminants. In particular, certain embodiments are directed to mutant bacteria for removal of heavy metals such as lead. In particular, bacterial mutant strains with increased endogenous cysteine synthesis provide efficient precipitation of soluble lead. A point of interest for the mutant lies in CC3625 cysteine synthase.

Throughout this patent document, various publications are referenced. The disclosures of these publications in their entireties are hereby incorporated by reference into this application in order to more fully describe the state of the art to which the disclosed matter pertains. While the following text may reference or exemplify specific factors or elements of a mutant or a method of utilizing the mutant, it is not intended to limit the scope of the invention to such particular reference or examples. Various modifications may be made by those skilled in the art, in view of practical and economic considerations, such as the surfaces or membranes for immobilizing the microorganisms and the suitable amount.

The articles “a” and “an” as used herein refers to “one or more” or “at least one,” unless otherwise indicated. That is, reference to any element or component of the present invention by the indefinite article “a” or “an” does not exclude the possibility that more than one of the element or component is present.

The term “about” as used herein refers to the referenced numeric indication plus or minus 10% of that referenced numeric indication.

The term “fluid” as used herein refers to a heavy-metal containing liquid. Examples include freshwater, waste water, organic and inorganic solvent that contain soluble lead.

The term “microorganisms” as used herein refers to bacteria, algae, yeasts, or fungi.

The term “mutagen” as used herein refers to a physical or chemical agent that can cause a change in the genetic material of the microorganisms. It includes radiation both ionizing or non-ionizing type as well as reactive chemicals such as alkaloids, reactive oxygen species, and alkylating agents or the like.

An aspect of the invention provides a mutant CC3625 cysteine synthase protein having an amino acid sequence that differs from SEQ ID NO: 1 by one or more of the following:
a) substitution of alanine in place of threonine at position 32,
b) substitution of valine in place of phenylalanine at position 101,
c) deletion of lysine at position 129, and
d) both substitution of threonine in place of methionine at position 271 and substitution of proline in place of serine at position 287.

The amino acid sequence for CC3625 cysteine synthase SEQ ID No: 1, is provided at GenBank: AAK25587.1

As further illustrated in the examples, CC3625 cysteine synthase is attributed to the precipitation of soluble lead. In some embodiments, the differences between the mutant CC3625 cysteine synthase protein and SEQ ID NO: 1 consists of 1, 2, 3 or 4 of the above a), b), c) and d). In some embodiments, the mutant CC3625 and SEQ ID NO: 1 differs additionally in other amino acid(s).

In some embodiments, the mutant CC3625 cysteine synthase has an amino acid sequence represented by SEQ ID NO: 2, which is also coded RCCR3 consisting of SEQ ID NO: 1 except substitution of alanine in place of threonine at position 32.

In some embodiments, the mutant CC3625 cysteine synthase has an amino acid sequence represented by SEQ ID NO: 3, which is also coded RCCR4 consisting of SEQ ID NO: 1 except substitution of valine in place of phenylalanine at position 101.

In some embodiments, the mutant CC3625 cysteine synthase has an amino acid sequence represented by SEQ ID NO: 4, which is also coded RCCR5 consisting of SEQ ID NO: 1 except deletion of lysine at position 129.

In some embodiments, the mutant CC3625 cysteine synthase has an amino acid sequence represented by SEQ ID No: 5, which is also coded RCCR7 consisting of SEQ ID No: 1 except both substitution of threonine in place of methionine at position 271 and substitution of proline in place of serine at position 287.

In some embodiments the mutant CC3625 cysteine synthase protein has an amino acid sequence consisting of one or more of SEQ ID Nos: 2, 3, 4, 5, and 11. Such sequences can further be carried with wild-type CC1482 translated amino acid sequence which is the enzyme catalyzing the first step of a multi-enzyme pathway in converting sulfate (SO₄⁻²) to H₂S, the substrate for the cysteine synthase enzyme. For example in one embodiment, RCCR3 carries SEQ ID Nos 2, 6 and wild-type CC1482; RCCR4 would carry SEQ ID Nos. 3, 6 and wild-type CC1482, RCCR5 carries SEQ ID Nos. 4, 6 and wild-type CC1482, and RCCR7 carries sequences 5, 6 and the wild-type CC1482.

In some embodiments the mutant CC3625 genotype has the nucleotide sequence of one or more of SEQ ID Nos: 12, 13, 14, and 15.

In some embodiments, the mutant CC1117 cysteine synthase protein has an amino acid sequence of one or more of SEQ ID Nos: 7, 8, 9, and 10. Such sequences can further be carried with wild-type CC1482 translated amino acid sequence or SEQ ID No. 11.

In some embodiments, the mutant CC1117 genotype has a nucleotide sequence of one or more of SEQ ID Nos: 17, 18, 19, and 20.

In another embodiment, the mutant CC1482 has an amino acid sequence represented by SEQ ID No: 11, which is also coded RCCR60.

In another embodiment the mutant CC1482 genotype has the nucleotide sequence of SEQ ID No. 21.

The invention also provides a fusion protein comprising one or more of the amino acid sequences disclosed herein. Exemplary embodiments include fusion proteins encoded by one or more sequences selected from SEQ ID No: 2, 3, 4, 5, and 11. Additional embodiments include fusion proteins encoded by one or more sequences selected from SEQ ID No: 12, 13, 14, and 15. Further embodiments include fusion proteins encoded by one or more sequences selected from SEQ ID NO. 7, SEQ ID No. 8, SEQ ID No. 9, and SEQ ID NO. 10. Further embodiments include fusion proteins encoded by one or more sequences selected from SEQ ID NO. 17, SEQ ID NO. 18, SEQ ID NO. 19, and SEQ ID NO. 20.

Various microorganisms may be modified to incorporate such a mutant CC3625, cysteine synthase protein or a mutant CC1117. A preferred embodiment is a bacterium, more preferably an oligotrophic fresh water bacterium such as Staphylococcus, Lactobacillus, Escherichia coli, Bifidobacteria, Bacteroides, Brevibacterium, and Caulobacter and a mutant thereof. In a further preferred embodiment, the bacterium is a Caulobacter crescentus strain. In comparison to a wild strain that expresses the CC3625 cysteine synthase having the amino acid sequence SEQ ID NO: 1, a Caulobacter crescentus strain with mutant CC3625 cysteine synthase is found to exhibit capacity to precipitate soluble lead. In another embodiment, a wild strain that expresses CC3625 cysteine synthase can be induced or programed to increase its capacity to precipitate soluble lead when cultured in a cysteine rich environment, where the effective cysteine concentrations ranging from 0.01 to 1000 μg/ml, preferably 0.05 to 750 μg/ml, and more preferably 0.10 to 500 μg/ml.

In some embodiment, the bacterial cell further contains a non-mutated gene CC1117. As illustrated in the examples, the LysR homolog encoded by CC1117 is believed to be related to the cysteine synthase activity. The CC1117 mutants described herein exhibit cysteine auxotrophy, suggesting that CC1117 is required for the expression of one or more genes required for cysteine biosynthesis. In some embodiments, the bacterial cell contains mutant CC 1117.

Another aspect of the invention provides a reactor for reducing the amount of soluble metal contaminants such as lead, mercury, zinc, copper, cadmium, manganese, chromium, cobalt, nickel, silver or arsenic in an aqueous fluid. In a preferred embodiment, the metal is lead. The reactor is constructed to contain a fluid conduit capable of contacting the fluid with the lead hyper-precipitating strain described herein.

The microorganisms may be immobilized on a surface of the fluid conduit by various means. Examples of the surface include, but are not limited to the surface of a pipe, the surface of a tank, the surface of a column-packing material, the surface of a screen, the surface of a porous filter substrate, the surface of replaceable cartridge and combinations of these.

Various shape or configuration of the surface can be used depending on the specific circumstance of the application. In some embodiments for example, a membrane can be adopted to incorporate microorganisms such as bacteria. The immobilized bacteria thereon and/or therein are alive or in a viable form and are liable to or effective to precipitate metals such as lead. The membranes can be of porous material, said material being either an inorganic oxide or a composite material containing an inorganic oxide and an organic polymer (e.g. a polysulfone), the membrane being such that the bacteria can settle in the pores, said pores communicating between themselves, so that it makes it possible an appropriate colonization of the membranes by the bacteria. In at least one embodiment, the membrane may be a cysteine rich membrane where the cysteine concentration is in the range of 0.01 to 1000 μg/ml, or 0.05 to 750 μg/ml, and preferably in the range of 0.10 to 500 μg/ml. An important feature of the membranes of the invention is the fact that the size of the pores is appropriate to the size of the bacteria which are immobilized in the membrane, i.e. large enough for the bacteria to settle and to grow and not too large to prevent the release of said microorganisms, especially in the fluid to be treated.

The bacteria may also be present in a biofilm on a surface of the fluid conduit. The biofilm can be a mass of bacteria which are fixed to each other by their own polymers. The biofilm results from the growth of the bacteria, which have previously been introduced into some pores on the surface of a membrane. The biofilm may also be formed in cracks of the skin side of the membrane, from the bacteria growing through the membrane. In some embodiments, the bacteria are in the form of a biofilm of a thickness of for example about 1 to about 200μ, all subranges and subunits included.

In some embodiments, the bacteria strain shows a high attaching capacity, which allows them to form aggregates of vegetative cells or spores and biofilms on a surface or solid support material. In some embodiments, the bacteria may be introduced in the form of spores. The spores may exist in a concentrated liquid suspension, a frozen concentrated suspension or a dehydrated form. The spores may be immobilized by support materials such as soil, sand, or polysaccharides (e.g. alginate, chitosan and agar) and other immobilizing agents. One or more accepted excipients, such as stabilizings, osmoprotectants or inhibitors, can also be used for stabilization purpose.

In some embodiments, the immobilized bacteria in the reactor are characterized by having a mutant CC3625 cysteine synthase selected from the group consisting of RCCR3 having amino acid sequence SEQ ID No: 2, RCCR4 having the amino acid sequence SEQ ID No: 3, RCCR5 having the amino acid sequence SEQ ID No: 4 and RCCR7 having the amino acid sequence SEQ ID No: 5.

In some embodiments, the reactor contains no exogenous cysteine. In some embodiments, exogenous cysteine can be introduced to facilitate the metal precipitation. The amount of the exogenous cysteine depends on the factors such as the bacteria and the condition of fluid to be treated. In one embodiment, the amount of cysteine can be in the ranges of 0.01 to 1000 μg/ml, between 0.05 to 750 μg/ml, preferably in the range of 0.10 to 500 μg/ml, or in such amounts as 50, 75, 100, 125, 150, 200, 250, 300, 400, and 500 μg/ml inclusive. One of ordinary skill in the art will be able to determine the suitable amount of exogenous cysteine without undue experimentation.

Another aspect of the invention provides a method of generating a lead hyper-precipitating strain of Caulobacter crescentus, the method comprising exposing bacteria of a first strain of Caulobacter crescentus to a mutagen, thereafter selecting exposed bacteria which exhibit greater cysteine synthase activity than do bacteria of the first strain that have not exposed to the mutagen, and culturing the selected bacteria to induce their proliferation, whereby the proliferated bacteria are the lead hyper-precipitating strain. Various methods of inducing mutation of bacteria are known in the art, including for example, Ely, et al., Transposon mutagenesis in C. crescentus. J. Bacteriol. 1982, 149: 620-625 and Ely, Genetics of Caulobacter crescentus. Methods Enzymol. 1991, 204: 372-384, the entire disclosure of which are hereby incorporated by reference.

Various methods for detecting cysteine synthase activity or endogenous cysteine synthesis are known in the art. Examples methods include measurement of cysteine as a red ninhydrin complex, derivatization of the thiol group of cysteine and identification by HPLC methods, determination of pyruvic acid after reacting cysteine with cysteine desulfhydrase, and application of protein binding assays. In addition, high throughput assays have also be used as in U.S. Pat. No. 6,605,459, the entire disclosure of which is incorporated by reference herein.

In some embodiments, the bacteria which exhibit greater cysteine synthase activity are selected by plating the proliferating bacteria on growth medium containing lead and selecting only cells that produce a visible brown color.

In some embodiments, the bacteria are originally auxotrophic for cysteine synthesis. However, the mutant bacteria exhibit greater cysteine synthase activity subsequent to selection on growth medium lacking cysteine.

In some embodiments, the bacteria are induced and trained to exhibit greater cysteine synthase activity when proliferating on a cysteine rich medium. The amount of cysteine can be in the ranges of 0.01 to 1000 μg/ml, between 0.05 to 750 μg/ml, preferably in the range of 0.10 to 500 μg/ml, or in such amounts as 50, 75, 100, 125, 150, 200, 250, 300, 400, and 500 μg/ml inclusive all intermediary measurements.

In some embodiments, this invention provides a method of generating a metal hyper-precipitating behavior in Caulobacter crescentus by exposing bacteria of a first strain of Caulobacter crescentus to a cysteine rich medium, wherein the medium contains a cysteine concentration ranging from about 0.01 to 1000 μg/ml, and thereafter selecting exposed bacteria which exhibit greater cysteine synthesis than do bacteria of the first strain, and culturing the selected bacteria to induce their proliferation, whereby the proliferated bacteria are the metal hyper-precipitating strain. In one embodiment, the metal is lead, mercury, zinc, copper, cadmium, manganese, chromium, cobalt, nickel, silver or arsenic. In a preferred embodiment, the metal is lead.

Another aspect of the invention provides a method of generating a lead hyper-precipitating strain of Caulobacter crescentus, the method comprising exposing bacteria of a first strain of Caulobacter crescentus to a mutagen thereafter selecting exposed bacteria which exhibit greater cysteine synthesis than do bacteria of the first strain that have not been exposed to the mutagen, and culturing the selected bacteria to induce their proliferation, whereby the proliferated bacteria are the lead hyper-precipitating strain.

Cysteine synthesis can be assessed by methods known in the art, including for example the procedure disclosed in J. Exp. Bot. (2000) 51 (347): 985-993, the entire discloure of which is incorporated by reference. Other assays that measures the synthesis of cysteine in whole cells or cell lysates are also applicable.

Another aspect of the invention provides a method of generating a lead hyper-precipitating strain of Caulobacter crescentus, the method comprising exposing bacteria of a first strain of Caulobacter crescentus to a mutagen culturing exposed bacteria in the presence of soluble lead, and thereafter selecting cultured bacteria which exhibit greater lead precipitation than do bacteria of the first strain that have not been exposed to the mutagen, whereby the selected bacteria are the lead hyper-precipitating strain.

In some embodiments, the lead precipitation is assessed by observing precipitation of lead hexaphosphate (LHP). In some embodiments, lead precipitation is assessed by observing formation of darker bacterial colonies on a solid growth medium including soluble lead. The lead precipitation may also be confirmed via x-ray diffraction. In some embodiments, lead precipitation is assessed by measuring the disappearance of removal of lead from surrounding media. Suitable methods include for example inductively couple plasma mass spectrometry (ICP-MS).

In some embodiments, to identify spontaneous mutants exhibiting increased lead precipitation activity, dilutions of overnight cultures comprising mutants can be spread onto a suitable growth medium, the selection of which can be determined by one of ordinary skill in the art without undue experimentation. Exemplary media include TYE (tryptone & yeast extract) and PYE (peptone & yeast extract). The lead source in the medium may comprise one or more compounds such as lead nitrate. Individual colonies are then screened for the brown color indicative of lead precipitation. While the entire brown colonies may not be observed via this procedure, colonies displaying a narrow brown sector can be observed at a certain frequency. A sterile needle can be used to remove cells from these brown sectors, and streaks on TYE+Pb(NO₃)₂ from these samples result in mixtures of entirely white and entirely brown colonies. Brown colonies from these streaks can then be picked to yield pure strains whose phenotype is uniformly brown on TYE+Pb(NO₃)₂ agar. The isolated strains can be further cultured to induce proliferation.

Another aspect of the invention provides a method of reducing the amount of soluble lead in an aqueous fluid, the method comprising contacting the fluid with the lead hyper-precipitating strain of Caulobacter crescentus of the present invention and thereafter separating the liquid and the strain.

In an exemplary embodiment, the method comprises: passing the fluid to be treated containing metal ions (e.g. lead, zinc, cadmium, nickel, copper) into the bioreactor; allow the fluid to contact to pre-implanted bacteria; repeating the above steps so that the metal ions are precipitated to a target level. As described, the bacteria may be immobilized on a surface of the fluid conduit of the reactor. If cells or spores are implanted on the surface, then a culture medium may be used to grow the bacteria to grow.

In some embodiments, the method further comprises isolation of the precipitated lead. Various known means of isolation can be used to recover the lead. Important steps of the isolation include for example, filtration/ separation, washing, and / or concentrating. In some embodiments, the lead is separated from the bacteria strain by cell lysis and separation of the precipitated lead from the insoluble fraction of the lysed cells.

Another aspect of the invention provides a method of synthesizing lead hexaphosphate, comprising contacting the fluid with the lead hyper-precipitating strain of Caulobacter crescentus of the present invention, and isolating the precipitated lead hexaphosphate.

### EXAMPLES

Bacterial strains and growth. All C. crescentus strains used in this study were derived from CB15 (ATCC 19089) or NA1000/CB15N as described in Table 1. All cultures were grown at 30° C. Liquid cultures were grown in PYE (peptone-yeast extract) medium (Poindexter J S. 1964. Biological properties and classification of the Caulobacter group. Bacteriol. Rev. 28: 231-295) or TYE (tryptone-yeast extract) medium (2 g tryptone, 1 g yeast extract, 0.2 g MgSO₄.7H₂O per liter filtered tap water, pH adjusted to 6.0). PYE agar (PYE medium plus 16 g/l agar), TYE agar (TYE medium plus 16 g/l agar) were used as solid complex media. When required, kanamycin was added to complex medium as follows: strains bearing Tn5 insertions 50 μg/ml in broth and agar; strains bearing CMS insertions 20 μg/ml in agar, 5 μg/ml in broth. Lead was added to complex medium in the range of 0.3 to 0.5 mM, because lead potency was affected by the quality of laboratory tap water. M2-glucose agar (Johnson R C, Ely B. 1977. Isolation of spontaneously derived mutants of Caulobacter crescentus. Genetics 86: 25-32.) was used as minimal medium for Caulobacter strains.

TABLE 1
Strains used in this study.^{a}

| Strain | Relevant genotype | Description or reference |
|---|---|---|
| CB15 | Wild-type (SEQ ID No. 22) | ATCC 19089 |
| CMS12 | NA1000 CC1029-1030::pBGS18T | 20^{c} |
| | (Kan^{R})^{b} | |
| CMS13 | NA1000 CC1107-CC1108::pBGS18T | 20^{c} |
| | (Kan^{R})^{b} | |
| CMS14 | NA1000 CC1189-CC1190::pBGS18T | 20^{c} |
| | (Kan^{R})^{b} | |
| CM539 | NA1000 CC3601-CC3603::pBGS18T | 20^{c} |
| | (Kan^{R})^{b} | |
| CMS40 | NA1000 CC3699-CC3700::pBGS18T | 20^{c} |
| | (Kan^{R})^{b} | |
| NA1000 | Cell cycle synchronizable | 21^{d} |
| | derivative of CB15 | |
| RCCR3 | hyp103 (SEQ ID No. 12) | spontaneous lead hyper- |
| | | precipitator |
| RCCR4 | hyp104 (SEQ ID No. 13) | spontaneous lead hyper- |
| | | precipitator |
| RCCR5 | hyp105 (SEQ ID No. 14) | spontaneous lead hyper- |
| | | precipitator |
| RCCR7 | hyp107 (SEQ ID No. 15) | spontaneous lead hyper- |
| | | precipitator |
| RCCR60 | hyp105 nop160 (SEQ ID No. 21) | UV-induced lead non- |
| | | precipitator |
| | | (pseudorevertant of |
| | | RCCR5) |
| RCCR61 | hyp105 nop161 | UV-induced lead non- |
| | | precipitator |
| | | (pseudorevertant of |
| | | RCCR5) |
| RCCR171 | CC3657::Tn5 hyp⁺ | φ(pooled random |
| | | Tn5) × RCCR4 → |
| | | Kan^{R} (Pb-white) |
| RCCR188 | hyp105 CC1070::Tn5~nop⁺ | φ(pooled random |
| | | Tn5) × RCCR61 → |
| | | Kan^{R} (Pb-brown) |
| RCCR194 | CC3657::Tn5 hyp104 | φ(RCCR171) × |
| | | RCCR4 → Kan^{R} |
| | | (Pb-brown) |
| RCCR261 | hyp103 nop261 (SEQ ID No. 17) | UV-induced lead non- |
| | | precipitator |
| | | (pseudorevertant of |
| | | RCCR3) |
| RCCR262 | hyp103 nop262 (SEQ ID No. 18) | UV-induced lead non- |
| | | precipitator |
| | | (pseudorevertant of |
| | | RCCR3) |
| RCCR263 | hyp103 nop263 (SEQ ID No. 19) | UV-induced lead non- |
| | | precipitator |
| | | (pseudorevertant of |
| | | RCCR3) |
| RCCR264 | hyp103 nop264 (SEQ ID No. 20) | UV-induced lead non- |
| | | precipitator |
| | | (pseudorevertant of |
| | | RCCR3) |
| RCCR302 | CC3657::Tn5 hyp104 | φ(RCCR194) × |
| | | NA1000 → KanR |
| | | (Pb-brown) |
| RCCR304 | CC3657::Tn5 hyp104 ΔphoY | φ(RCCR194) × |
| | | YJ10010 → Kan^{R} |
| | | (Pb-brown) |
| YJ10010 | NA1000 ΔphoY | 19^{e} |

^{a}Kan^{R}, resistance to kanamycin; Pb-brown and Pb-white, brown and white colony color, respectively, on solid medium containing lead nitrate.
^{b}Open reading frame annotations same as those used in original publication and annotation of CB15 genome (Nierman WC, Feldblyum TV, Laub MT, Paulsen IT, Nelson KE, Eisen J, Heidelberg JF, Alley MR, Ohta N, Maddock JR, Potocka I, Nelson WC, Newton A, Stephens C, Phadke ND, Ely B, DeBoy RT, Dodson RJ, Durkin AS, Gwinn ML, Haft DH, Kolonay JF, Smit J, Craven MB, Khouri H, Shetty J, Berry K, Utterback T, Tran K, Wolf A, Vamathevan J, Ermolaeva M, White O, Salzberg SL, Venter JC, Shapiro L, Fraser CM. 2001. Complete genome sequence of Caulobacter crescentus. Proc. Natl. Acad. Sci. USA 98: 4136-4141) rather than current NA1000 annotation.
^{c}West L, Yang D, Stephens C. 2002. Use of the Caulobacter crescentus genome sequence to develop a method for systematic genetic mapping. J. Bacteriol. 184: 2155-2166.
^{d}Evinger M, Agabian N. 1977. Envelope-associated nucleoid from Caulobacter crescentus stalked and swarmer cells. J. Bacteriol. 132: 294-301.
^{e}Yung MC, Jiao Y. 2014. Biomineralization of uranium by PhoY phosphatase activity aids cell survival in Caulobacter crescentus. Appl. Envion. Microbiol. 80: 4795-4804. http://dx.doi.org/10.1128/AEM.01050-14

Isolation of mutants. To identify spontaneous mutants exhibiting increased lead precipitation activity, dilutions of overnight cultures of CB 15 were spread onto TYE agar containing 0.5 mM lead nitrate and individual colonies were screened for the brown color indicative of lead precipitation. Entire brown colonies were not observed via this procedure, but colonies displaying a narrow brown sector were observed at a frequency of approximately 1×10⁻⁵. A sterile needle was used to remove cells from these brown sectors, and streaks on TYE+Pb(NO₃)₂ from these samples resulted in mixtures of entirely white and entirely brown colonies. Brown colonies from these streaks were picked to yield pure strains whose phenotype was uniformly brown on TYE+Pb(NO₃)₂ agar. Non-precipitating suppressor mutants were isolated via UV mutagenesis of hyper-precipitating strains. Cells were irradiated to 10⁻¹ survival and then plated onto TYE+Pb(NO₃)₂ agar. The resulting colonies were screened for white coloration. White colonies appeared at a frequency of approximately 2×10⁻⁵.

Preparation of lead phosphate precipitate. A flask containing 1 liter of TYE broth containing 0.05 mM lead nitrate was inoculated with 1 ml of an overnight TYE broth culture of C. crescentus strain RCCR3. Preparation of the brown lead precipitate was carried out as described previously (Mire C E, Tourjee J A, O'Brien W F, Ramanujachary K V, Hecht G B. 2004. Lead precipitation by Vibrio harveyi: Evidence for novel quorum sensing interactions. Appl. & Environ. Microbiol. 70: 855-864.), with the exception of substituting N-lauroyl-sarcosine for sodium dodecyl sulfate. Analysis and identification of the lead phosphate precipitate by x-ray diffraction was carried out as described previously, and the resulting pattern was identified by using the JCPDS library of compounds.

Genetic manipulations of bacterial strains. Random Tn5 mutagenesis was carried out as described previously (Ely B, Croft R H. 1982. Transposon mutagenesis in C. crescentus. J. Bacteriol. 149: 620-625; Ely B. 1991. Genetics of Caulobacter crescentus. Methods Enzymol. 204: 372-384). Generalized transductions using φCr30 were carried out as described previously (Ely B, Johnson R C. 1977. Generalized transduction in Caulobacter crescentus. Genetics 87: 391-399.) for both genetic mapping and strain construction experiments.

Inverse PCR and sequencing of PCR products. Identification of the Tn5 insertion sites in RCCR171 and RCCR188 was carried out by inverse PCR as described previously (Brun Y V, Shapiro L. 1992. A temporally controlled sigma-factor is required for polar morphogenesis and normal cell division in Caulobacter. Genes Dev. 6: 2395-2408.). Chromosomal DNA for PCR was prepared using a DNeasy Blood & Tissue Kit (Qiagen, Valencia Calif.). PstI restriction digests, ligations, and other DNA manipulations were carried out as described previously (Sambrook J, Fritsch E F, Maniatis T. 1989. Molecular cloning: A laboratory manual. Cold Spring Harbor Press, Cold Spring Harbor, N.Y.). Oligonucleotide primers R3 and L7, which specifically anneal to Tn5 sequences, were prepared by Eurofins MWG Operon (Huntsville Ala.). Sanger sequencing of the PCR amplification products was carried out by Eurofins MWG Operon (Huntsville Ala.).

Whole genome sequencing. DNA was isolated from overnight cultures using the Gentra Puregene Yeast/Bacteria kit (Qiagen). Twelve multiplex libraries were made using the IntegenX Apollo 324™ System. The libraries were sequenced on a single lane of an Illumina HiSeq, to a read length of 140 bases, resulting in an average fold-coverage of 400 for each strain. The reads were mapped to the C. crescentus CB15 genome (NCBI NC_002696.2) using BWA for Illumina (Li H, Durbin R. 2009. Fast and accurate short read alignment with Burrows-Wheeler Transform. Bioinformatics 25:1754-60.). The resulting SAM files were converted to BAM files using SAMtools (Li H, Handsaker B, Wysoker A, Fennell T, Ruan J, Homer N, Marth G, Abecasis G, Durbin R, 1000 Genome Project Data Processing Subgroup. 2009. The Sequence alignment/map (SAM) format and SAMtools. Bioinformatics 25:2078-9). The BAM files were visualized using Integrated Genome Viewer (IGV) (Robinson J T, Thorvaldsdóttir H, Winckler W, Guttman M, Lander E S, Getz G, Mesirov J P. 2011. Integrative Genomics Viewer. Nature Biotechnology 29:24-26.), manually searching the genomes for SNPs, deletions and amplifications. All mutations are described in Table 1. Our laboratory CB15 strain contained mutations relative to the reference CB 15 genome (NCBI NC_002696.2) and those mutations are also noted in Table 1.

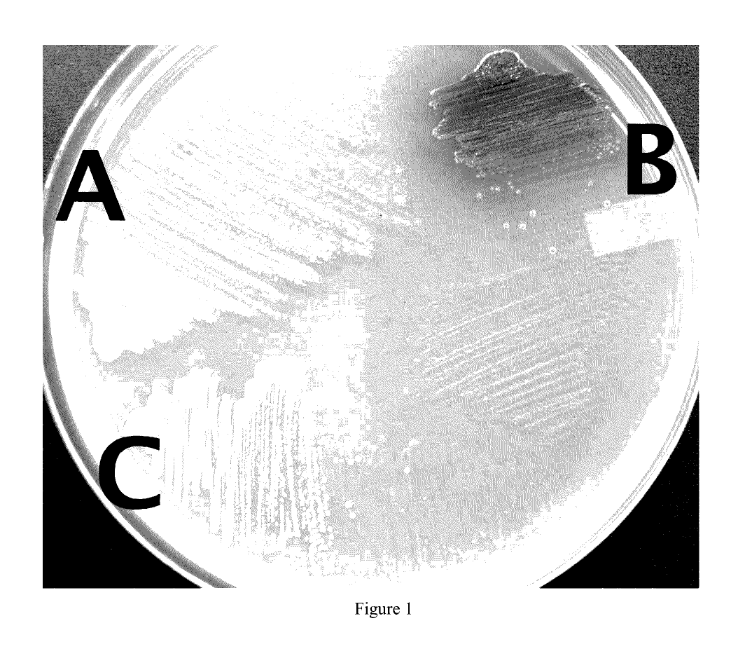

RESULTS: Isolation of Pb₉(PO₄)₆ hyper-precipitation mutants. Colonies of CB15 are normally off-white in color, even in the presence of lead nitrate, indicating that wild-type Caulobacter is not an efficient precipitator of lead nitrate. Four independent lead hyper-precipitator mutants isolated as described in the Materials and Methods were chosen for further study. These strains, each of which displays a brown colony color when grown on solid medium containing lead nitrate, were designated as hyp (hyper-precipitator) strains. FIG. 1 illustrates the lead precipitation effect of a mutant strain in comparison with a wild-type strain.

The brown precipitate formed by one of these mutants, RCCR3, was purified as described in the Materials and Methods. The x-ray diffraction pattern of the precipitate matches closely the pattern reported for JCPDS card no. 33-0768 and thus appears to be identical to the Pb₉(PO₄)₆ compound precipitated by Vibrio harveyi. It was concluded that the precipitate formed by the Caulobacter mutants is Pb₉(PO₄)₆.

Transductional mapping of the hyp mutations. Experiments were carried out to isolate a Tn5 linked to the hyp locus in each of the four mutant strains. Bacteriophage φCr30 lysates were prepared on pooled cultures of random Tn5 insertions in CB 15 prepared as described previously. These lysates were then used as donors in generalized transduction crosses in which the individual hyp strains served as recipients. Transductants were plated onto TYE containing both kanamycin and lead nitrate. The kanamycin served as a selectable marker for cells that had a successful recombination event with a DNA fragment bearing a Tn5 element from the φCr30 lysate. The presence of lead nitrate in the medium allowed for immediate color screening of transductant colonies to determine their lead precipitation phenotype. White colonies arising in this experiment are expected to be the result of replacement of the mutant hyp locus with the wild-type hyp⁺ allele linked to Tn5. Strain RCCR171 (Tn5˜hyp⁺) was constructed in this manner from the hyp104 strain RCCR4 (see also Table 1).

The linkage of the Tn5 in RCCR171 to the hyp locus was verified by backcrossing a φCr30 lysate of RCCR171 into the parental hyp104 strain. Crossing φCr30 RCCR171)×RCCR4 would be expected to yield a mixture of Tn5˜hyp104 (brown) and Tn5˜hyp⁺ (white) colonies on medium containing lead nitrate. Indeed, this cross gave approximately 70% brown (hyp) and 30% white (hyp⁺) transductants. One of the brown colonies generated by this cross (Tn5˜hyp104) was saved as RCCR194 and used as a donor in a transductional cross with CB15. In this cross, approximately 30% of the transductants displayed the hyper-precipitation phenotype and 70% retained the wild-type phenotype. Taken together, these crosses indicate that a single locus is responsible for the lead hyper-precipitation phenotype in RCCR4, and that this same locus is sufficient to cause the hyper-precipitation phenotype.

The same set of experiments was carried out to obtain Tn5 insertions linked to the hyp103 and hyp105 mutations, all of which generated similar results (data not shown). Additional crosses with the various Tn5˜hyp⁺ insertions indicated that the hyp103, hyp104, and hyp105 mutations all belong to a single transductional linkage group (data not shown). To identify the region containing the hyp locus, inverse PCR (Brun Y V, Shapiro L. 1992. A temporally controlled sigma-factor is required for polar morphogenesis and normal cell division in Caulobacter. Genes Dev. 6: 2395-2408) was carried out on chromosomal DNA from RCCR171. Sequence analysis of the amplification product revealed the site of the Tn5 insertion to be within gene CC3657.

Additional experiments were performed using the integrated plasmid markers from strains CMS39 and CMS40. The CC3657 gene lies between the map locations of these particular markers. The insertions within CMS39 and CMS40 were used as markers in transduction experiments to (a) convert the hyp allele to wild-type, and (b) introduce the hyp allele into CB 15. These crosses confirmed the conclusions regarding the general location of the hyp locus and that the identified hyp locus is both necessary and sufficient to confer the observed hyper-precipitation phenotype in RCCR3, RCCR4, and RCCR5.

Tn5 insertions linked to the hyp locus in RCCR7 could not be identified because transductions using RCCR7 as a recipient did not yield any transductants. It was verified that RCCR7 can support successful φCr30 infection, but the reason for its inability to produce transductants is unclear.

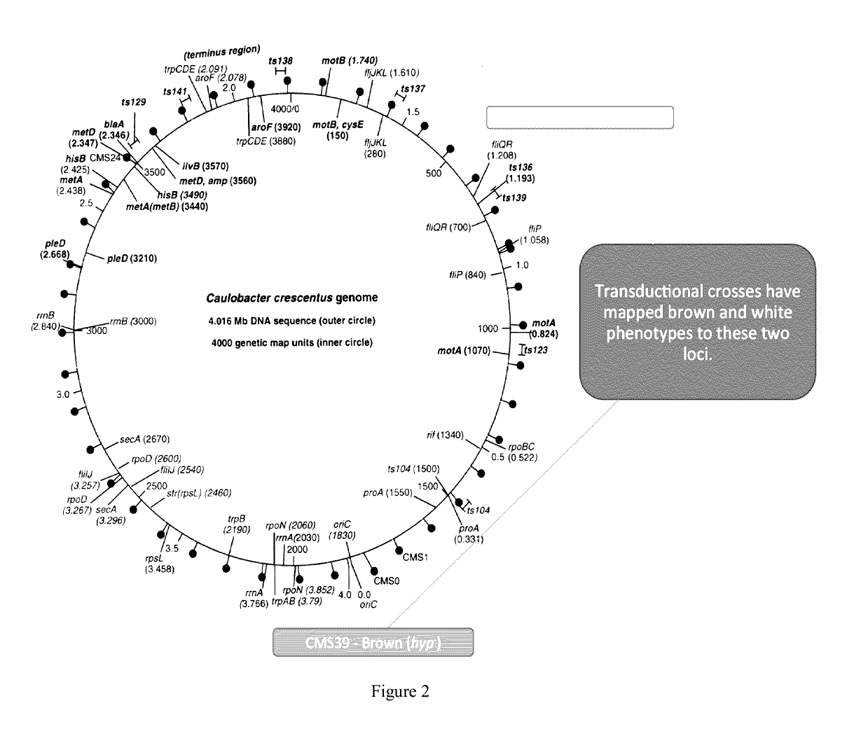

FIG. 2 illustrates the transductional mapping of the C. crescentus genome. A locus has been identified which is linked with the brown hyp phenotype. A separate locus linked with the white nop phenotype has also been identified.

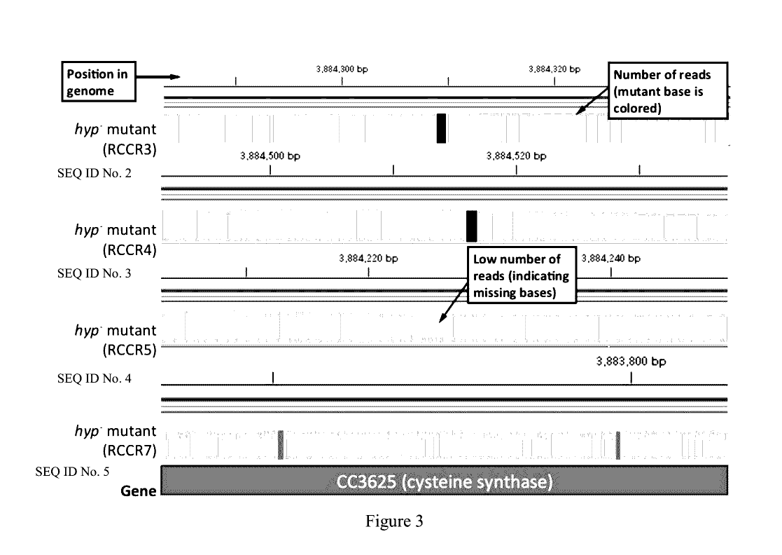

FIG. 3 illustrates each of the brown hyp mutants for CC3625. This suggests that this gene is necessary and sufficient for the brown lead precipitating phenotyple.

Isolation and mapping of non-precipitation suppressors. To identify additional loci that play an important role in the process of lead precipitation, the experiment was set for identifying non-precipitating suppressor mutations of the hyper-precipitation phenotype. Attempts at isolating spontaneous non-precipitator pseudorevertants were not successful. Consequently, UV mutagenesis was employed as described in the Materials and Methods to generate a collection of pseudorevertants from the RCCR3 and RCCR5 hyper-precipitation strains. The suppressor mutations carried by these strains were designated nop (non-precipitator).

Using the same strategies as outlined for hyp mutants, a Tn5 insertion linked to the nop161 mutation in strain RCCR61 (hyp105 nop161) was obtained to generate strain RCCR188 (hyp105 Tn5˜nop⁺). Inverse PCR was performed on RCCR188, and DNA sequence analysis of the amplification product identified the Tn5 insertion site to be within open reading frame (ORF) CC1070. Transductions using RCCR188 as a donor demonstrated that the CC1070::Tn5 could be used to recombine out the nop261, nop262, nop263, and nop264 alleles, placing those mutations into the same linkage group as nop161. Additional transductions were performed with the integrated markers in CM12, CMS13 and CMS14 as the map locations of these markers surround the CC1070 locus. These crosses confirmed that the locus for these nop mutations is linked to the CMS13 and CMS14 marker loci but not the CMS12 locus. Thus, the nop locus is linked the region between CC1107 and CC1190, clearly distinct from the hyp locus characterized above. Further, crosses between the nop-linked CC1070::Tn5, CMS13, and CMS14 markers linked to the nop⁺ allele and all hyp nop strains resulted in a mixture of hyper- and non-precipitator transductants, demonstrating that the nop locus influences precipitation independently of the hyp locus and is necessary to confer the suppression of the hyper-precipitation phenotype.

The CC1070::Tn5 was also used to backcross the nop261, nop262, nop263, and nop264 alleles into RCCR3 via transduction to construct hyp103 Tn5˜nop strains. As expected, these strains all exhibit a non-precipitation phenotype. Taken together, the experiments described here demonstrate that the nop locus linked to CC1070 is sufficient to suppress the hyper-precipitation phenotype expressed by the hyp locus linked to CC3657.

Identification of hyp and nop mutations by genomic sequencing. The experiments described above identified the general genomic locations of the causative hyp and nop mutations. To identify the specific hyp and nop mutation sites, the genomes of parental CB 15, brown mutants (RCCR3, RCCR4, RCCR5 and RCCR7) and white mutants (RCCR261, RCCR262, RCCR263 and RCCR264) were sequenced. All of the mutations (SNPs, insertions and deletions) in these strains were identified and compiled in Table 1. The hyp mutations were expected to have two properties: (1) be present in both the hyp and subsequent hyp nop mutants but not in CB15, and (2) be linked to CC3656 (as determined by the above transduction analysis). All four hyp strains contain mutations in the ORF of CC3625 (encoding cysteine synthase) that are consistent with these criteria. Three of the strains contain at least one missense mutation (RCCR3: F101V; RCCR4: T32A; RCCR7: M271T and S287P), while one strain contains a 3-bp deletion that results in a single-amino acid deletion (RCCR5: L129del). Overlaying these mutations on a sequence alignment of CC3625 and other cysteine synthases shows that all hyp strains contain mutations in highly conserved residues. Specifically, the mutation in RCCR3 is, as expected, present in all hyp nop strains, which are derived from RCCR3.

The nop mutations were expected to have two properties: (1) be present in the hyp nop mutants but not in the hyp mutants or in CB15, and (2) be linked to CC 1070 (as determined by the above transduction analysis). All four hyp nop strains contain mutations in the ORF of CC1117 (a LysR-family transcription factor) that are consistent with these criteria. One of the strains contains a frameshift mutation (RCCR263: R25 Vs), suggesting that a loss of CC1117 function causes the nop phenotype. The three other strains contain missense mutations (RCCR261: S255F; RCCR262: E183K; RCCR264: G249A). Overlaying these mutations on a sequence alignment of CC1117 and other LysR homologs shows that the mutations affect conserved residues in the substrate domain. In summary, these sequencing results, coupled with the transduction mapping data, indicate that the hyp phenotype is caused by mutations in CC3625 and the nop phenotype is caused by mutations in CC1117.

Increased cysteine levels promote lead precipitation. The observation that mutations in CC3625 confer a hyper-precipitation phenotype suggested that altered levels of cysteine promote lead precipitation. To test the effect of increased cysteine, several strains from this study were grown on PYE medium supplemented with both lead nitrate and cysteine. The CB 15 and RCCR263 strains, neither of which normally precipitate lead, both produced visible Pb₉(PO₄)₆ in the presence of exogenous cysteine. This result is consistent with the hypothesis that the lead precipitation phenotype of hyp mutants is the consequence of increased endogenous cysteine synthesis caused by gain-of-function mutations in CC3625.

LysR-type regulators often control expression of amino acid synthesis genes (Wendish, 2007; Book on amino acid biosynthesis). Because the nop mutations all lie within CC1117, encoding a LysR family member, it was predicted that the nop mutants are not able to precipitate lead because of an inability to synthesize cysteine. To test this, strains from this study were grown on minimal M2 plates lacking cysteine. CB 15 and hyper-precipitating strains formed colonies on M2, but strains bearing a nop mutation did not. In contrast, M2 medium supplemented with cysteine was able to support growth of nop strains. These results support the hypothesis that the LysR homolog encoded by CC1117 is required for cysteine synthase activity.

Sequence analysis of CC1117. Our results suggest that the transcription factor CC1117 is responsible for activating genes required for cysteine biosynthesis in C. crescentus. In E. coli, this function is carried out by CysB, a member of the LysR family of transcription factors that respond to nutritional and environmental stimuli. CC1117 is also in the LysR family and the test was conducted to determine whether CC1117 is related to CysB. LysR proteins consist of two domains, an N-terminal DNA-binding domain and a C-terminal substrate domain. CysB has a specific substrate domain that places it into the CysB sub-family. To determine the sub-family containing CC1117, the substrate domains of LysR proteins from many species were compared using multiple sequence alignment. Note that pairs of proteins were selected from the same set of species to prevent bias due to evolutionary distance. As such it was found that the substrate domain of CC1117 most closely matches domains within the GcvA sub-family, but is more distantly related to the CysB sub-family and other LysR homologs, C. crescentus CC1621 and E. coli LysR. In contrast, the substrate domain of E. coli CysB most closely matches domains within the CysB sub-family. The percentage identities between all of these domains were used to make an average distance tree, which supports the conclusion that CC1117 is in the GcvA sub-family and not in the CysB sub-family. In addition, the structure of K. aerogenes CysB in complex with a substrate has been solved (PDB 1AL3; ref 35) and shows 7 residues lining the substrate pocket. These residues are conserved in CysB orthologs but not in other LysR homologs, including CC1117.

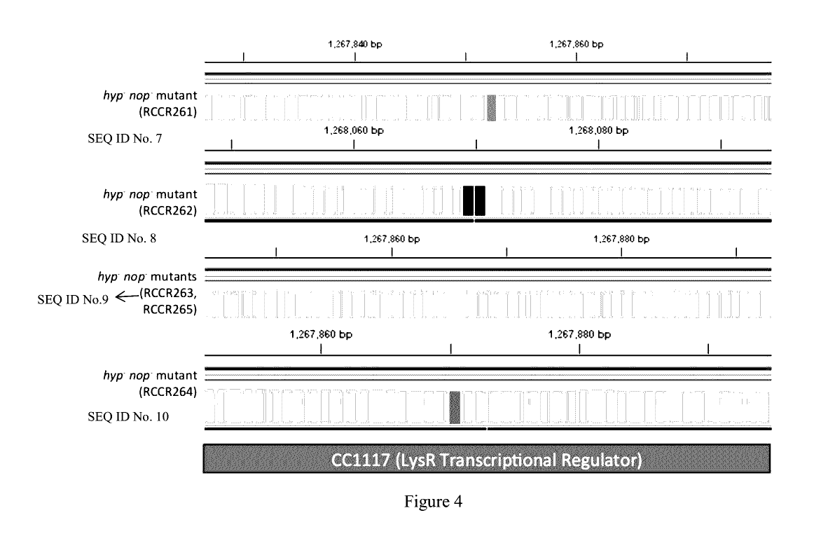

FIG. 4 illustrates five of the white hyp nop strains having a mutation in CC1117. This suggests that loss of function of this gene is sufficient for suppressing the brown lead precipitation phenotyple.

It is confirmed that C. crescentus CC1117 and E. coli GcvA are orthologs using reciprocal BLAST analysis (36). BLAST of the E. coli genome using the entire CC1117 sequence as a query resulted in GcvA as the best hit (query coverage 89%, E=4×10⁻⁷⁷), while BLAST of the C. crescentus genome using GcvA as a query resulted in CC1117 as the best hit (query coverage 93%, E=1×10⁻⁷⁶). However, E. coli CysB does not appear to have a C. crescentus ortholog. BLAST of the C. crescentus genome using CysB as a query resulted in CC1621 as the best hit, but BLAST of the E. coli genome using CC1621 as a query resulted in LysR as the best hit. Taken together, our sequence analysis indicates that CC1117 is a GcvA ortholog and the C. crescentus genome does not contain a CysB ortholog.

Deletion of phoY does not suppress lead phosphate precipitation. The ability of Caulobacter to biomineralize heavy metals to insoluble phosphate salts is not restricted to lead. The organism is able to tolerate high concentrations of uranium, a consequence of the periplasmic alkaline phosphatase PhoY. Moreover, over-expression of phosphatase enzymes is apparently sufficient to confer uranium precipitation and biosorption by microorganisms. Phosphatase activity has also been shown to be involved in the precipitation of lead phosphate salts by Cupriavidus metallidurans and specifically Pb₉(PO₄)₆ by Providencia (10). Thus, it would not be surprising if phosphatase activity were required for Pb₉(PO₄)₆ precipitation in Caulobacter. Interestingly, no mutations in genes encoding PhoY or other phosphatase enzymes were observed in any of the mutants in our collection.

To test whether PhoY is required for lead phosphate precipitation, RCCR194 was used as a transductional donor to introduce the hyp104 allele into the ΔphoY strain YJ10010. The transductants arising from this cross were screened for their lead precipitation phenotype, and a number of these were in fact positive for lead precipitation. Lead precipitation by YJ10010 was also observed when the strain was grown on solid medium containing lead and excess cysteine. It was concluded that PhoY is not required for precipitation of Pb₉(PO₄)₆ by Caulobacter.

Discussion. The experiments reported here demonstrate that cysteine plays an important role in the precipitation of Pb₉(PO₄)₆ by Caulobacter crescentus. Genetic analysis of hyper-precipitating strains RCCR3, RCCR4, RCCR5 and RCCR7 reveals that mutations in the cysteine synthase gene CC3625 are both necessary and sufficient to confer the lead hyper-precipitation phenotype of those strains. High concentrations of exogenous cysteine can enable strains that are normally poor lead precipitators to become more efficient precipitators. Whether the exogenous cysteine in the experiments enters the cytoplasm, only enters the periplasm, or somehow works only at the outer surface of the cell is under investigation. Regardless, the observation that exogenous cysteine promotes precipitation activity is consistent with the hypothesis that increased cysteine synthase activity causes the hyper-precipitation phenotype.

Analysis of the C. crescentus genome reveals two paralogous cysteine synthase genes, CC3625 and CC1426. However, the hyp mutations were only found in the CC3625 gene, suggesting that increased activity of the CC3625 enzyme, but not the CC1426 enzyme, is able to promote lead precipitation. Importantly, E. coli also contains two cysteine synthase genes; it was found through reciprocal BLAST analysis that CC3625 is orthologous to cysK and CC1426 is orthologous to cysM. However, it has been suggested that CysM is not important for cysteine biosynthesis since deleting cysK, but not cysM, causes cysteine auxotrophy. Whether CC1426 participates in C. crescentus cysteine biosynthesis is to be determined.

Genetic analysis of non-precipitating pseudorevertants indicates that mutations in CC1117, encoding a transcription factor of the LysR family, are able to suppress the hyper-precipitation phenotype that is caused by mutations in CC3625. The observation that one of the nop mutants in our collection contains a frameshift argues strongly that a loss of CC1117 function is responsible for the suppression phenotype. Additionally, the CC1117 mutants described here exhibit cysteine auxotrophy, suggesting that CC1117 is required for the expression of one or more genes required for cysteine biosynthesis. The fact that CC1117 regulates cysteine metabolism was unexpected because it was found that CC1117 is an ortholog of E. coli GcvA, a regulator of operons involved in the glycine cleavage system. Neither GcvA nor its orthologs have been shown to regulate cysteine metabolism. Thus, CC1117 is the first member of the GcvA sub-family to be implicated in regulating cysteine metabolism. Although not comprehensive, there are 141 GcvA homologs listed in the NCBI Conserved Domains database, in the category entitled “LysR-type GcdR, TrPI, HvR and beta-lactamase regulators”. While a few of these have been shown to regulate different aspects of metabolism, the vast majority has not been characterized. It will be important to understand how these seemingly similar transcription factors have acquired many different functions.

It was found that CC1117 is not orthologous to E. coli CysB, a known activator of the cysteine regulon in that organism. Other bacterial species, like Aspergillus nidulans and Pseudomonas putida, have been shown by sequence and functional analysis to contain CysB orthologs. Some potential CysB orthologs have been identified by sequence analysis (NCBI Conserved Domains) but have yet to be tested for function. However, C. crescentus, like some other bacterial species, does not appear to have any CysB ortholog. It appears that bacteria use an array of mechanisms to regulate cysteine metabolism. In Bacillus subtilis, CymR is a Rrf2 family transcription factor and represses cysteine metabolism genes. In Lactococcus lactis, FhuR is a LysR family member related to E. coli OxyR and regulates genes required for general sulfur amino acid metabolism. In Corynebacterium glutamicum, McbR, a transcriptional repressor in the TetR family also regulates genes involved in sulfur amino acid metabolism. Thus, it appears that CC1117 is just one of many types of transcription factors that regulate cysteine metabolism in prokaryotes.

Implications for the mechanism of lead phosphate precipitation. The mechanistic relationship between cysteine availability and the actual synthesis of Pb₉(PO₄)₆ is not immediately apparent from the experiments described here. However, cysteine has been broadly associated with tolerance to heavy metals. For example, the cysK gene is known to be important in tellurite reduction and resistance in Rhodobacter sphaeroides, and cysM mutations confer tellurite sensitivity in Staphylococcus aureus. In Geobacter stearothermophilus, multiple components of the cysteine metabolic pathway are involved in tellurite resistance, including CysK, the cysteine desulfurase IscS, and the siroheme synthase CobA; in addition, cysteine metabolism genes are up-regulated in response to tellurite exposure. Enterobacter sp. YSU, normally sensitive to selenite on minimal medium, exhibits resistance in the presence of exogenous cysteine. Transgenic tobacco plants overexpressing cysteine synthase display increased tolerance to a number of heavy metals. Thus, the implication that cysteine is important in Caulobacter lead precipitation fits well with the widespread role of cysteine in the cellular response to heavy metals.

There are many models, not necessarily mutually exclusive, that explain the role of cysteine in lead precipitation. Cysteine can be biochemically converted to glutathione, and both glutathione and cysteine serve as cellular antioxidants. Lead is a strong oxidant and can cause cellular damage by forming free radicals. One possibility is that reduction or binding of lead by cysteine and/or glutathione is necessary for one of the steps that ultimately yields Pb₉(PO₄)₆. Alternatively, lead may cause damage to a cellular component that is required for the lead precipitation reaction, and sufficiently high levels of cysteine and/or glutathione reverse this damage. Another possible model is that cysteine and/or glutathione might serve to reduce and subsequently activate a cellular molecule that promotes lead precipitation. Additional work will be necessary to distinguish between these and other models.

Phosphatases are believed to be important for lead precipitation in some microorganisms by increasing the local pool of inorganic phosphate that is available to react with free metal ions. The resulting lead phosphate salts are insoluble, thus impeding the ability of the metal to diffuse through the aqueous cellular environment and damage biomolecules. A similar role has been proposed for the PhoY phosphatase in uranium precipitation by Caulobacter. The observation that in PYE medium the same ΔphoY allele can suppress precipitation of uranium but not lead suggests that the cellular pathways for mineralizing the two metals are not identical. The results reported here suggest that the decreased phosphate availability attributable to ΔphoY is not sufficient to prevent lead precipitation in plate assays. It remains possible that phosphatase activity is required for lead precipitation by Caulobacter; indeed, there are several phosphatase genes in the Caulobacter genome that may play a role. While PhoY is required for uranium precipitation in Caulobacter, it does not appear that the high concentration of cysteine required for lead precipitation in this study is necessary for uranium precipitation, nor does exposure to uranium appear to increase expression of cysteine synthase. Whether less or no cysteine is important for uranium precipitation remains to be seen. Interestingly, at least two aspects of the Caulobacter precipitation mechanism are different between uranium and lead.

In present invention, inventors describe the isolation of a number of Caulobacter mutants that form colonies readily in the presence of lead nitrate and also mineralize Pb₉(PO₄)₆. The results presented here are consistent with a model in which lead mineralization by Caulobacter requires high levels of either intracellular or exogenous cysteine. Interestingly, the phosphate necessary for lead precipitation can be derived even in the absence of PhoY, a phosphatase required for uranium precipitation. These results strongly suggest that Caulobacter employs at least two different mineralization processes. Our results, coupled with the fact that Caulobacter can readily form biofilms, indicate that Caulobacter can be used for lead biosorption applications.

In another example, a test was conducted to ascertain an effective amount of the exogenous cysteine in the growth medium for purposes of inducing lead precipitation by the wild-type Caulobacter strain, Caulobacter crescentus strains were grown at 30° C. for five days on PYE medium containing 0.3 mM Pb(NO₃)₂ (left) and PYE medium containing 0.3 mM Pb(NO₃)₂ and 125 μg/ml cysteine (right). The contents of PYE medium per liter are as follows: 2.0 g peptone, 1.0 g yeast extract, 0.2 g MgSO₄.7H₂O, pH =6.0; solvent is filtered tap water rather than distilled water.

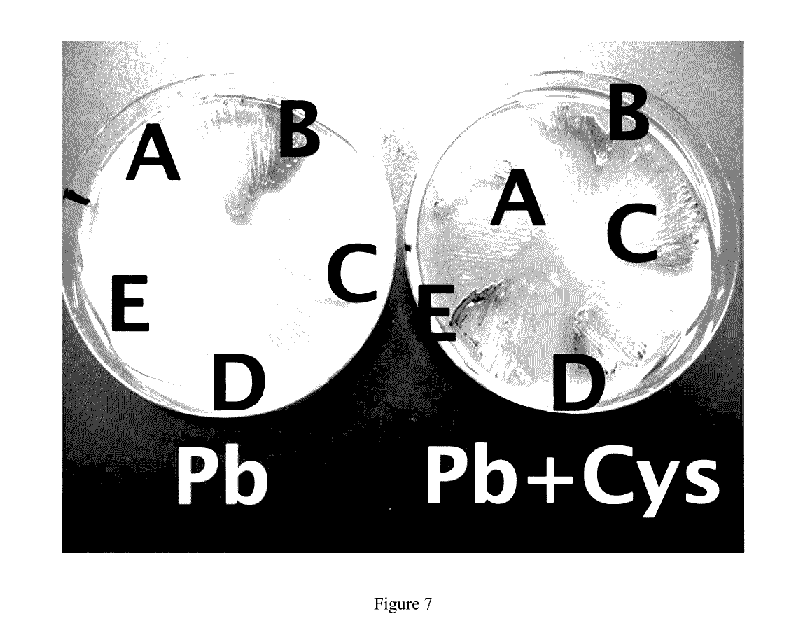

FIG. 7 illustrates the lead precipitation effect of several different Caulobacter strains in comparison with a wild-type strain. The wild-type CB 15 is shown in (A), the hyper-precipitator RCCR3 (B), the non-precipitator RCCR263 (C), NA1000 (D), and the NA1000 ΔphoY deletion strain YJ10010 (E).

Those of ordinary skill in the art can appreciate that high cysteine concentrations in the medium enhanced the bacterial capabilities to precipitate lead. This is evident because strains that do not have mutations in the CC3625 gene (such as strains CB15, NA1000, YJ10010) can be induced to precipitate lead when grown in the presence of exogenous cysteine. Moreover, the presence of a nop mutation (such as strain RCCR263) does not prevent exogenous cysteine from inducing lead precipitation.

It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A mutant CC3625 cysteine synthase protein having an amino acid sequence that differs from SEQ ID NO: 1 in a way selected from the group consisting of
a) substitution of alanine in place of threonine at position 32,
b) substitution of valine in place of phenylalanine at position 101,
c) deletion of lysine at position 129, and
d) both substitution of threonine in place of methionine at position 271 and substitution of proline in place of serine at position 287,
the mutant CC3625 cysteine synthase protein conferring to a Caulobacter crescentus strain that expresses it a greater capacity to precipitate soluble lead than a strain that expresses the CC3625 cysteine synthase having the amino acid sequence SEQ ID NO: 1.

2. The mutant CC3625 cysteine synthase protein of claim 1 selected from the group consisting of
a) RCCR3, having the amino acid sequence SEQ ID NO: 2,
b) RCCR4, having the amino acid sequence SEQ ID NO: 3,
c) RCCR5, having the amino acid sequence SEQ ID NO: 4, and
d) RCCR7, having the amino acid sequence SEQ ID NO: 5.

3. The mutant CC3625 cysteine synthase of claim 2, being RCCR3, having the SEQ ID NO: 12.

4. The mutant CC3625 cysteine synthase of claim 2, being RCCR4. having the SEQ ID NO: 13.

5. The mutant CC3625 cysteine synthase of claim 2, being RCCR5, having the SEQ ID NO: 14.

6. The mutant CC3625 cysteine synthase of claim 2, being RCCR7, having the SEQ ID NO: 15.

7. A lead hyper-precipitating strain of Caulobacter crescentus that expresses the mutant CC3625 cysteine synthase of claim 1.

8. A reactor for reducing the amount of soluble lead in an aqueous fluid, the reactor comprising a fluid conduit capable of contacting the fluid with the lead hyper-precipitating strain of claim 7.

9. The reactor of claim 8, wherein the lead hyper-precipitating strain is immobilized on a surface of the fluid conduit.

10. The reactor of claim 8, wherein the lead hyper-precipitating strain is present in a biofilm on a surface of the fluid conduit.

11. The reactor of claim 9, wherein the surface is selected from the group consisting of the surface of a pipe, the surface of a tank, the surface of a column-packing material, the surface of a screen, the surface of a porous filter substrate, and combinations of these.

12. The reactor of claim 8, wherein the lead hyper-precipitating strain is immobilized on a surface of replaceable cartridge which contacts fluid in the fluid conduit.

13. A method of generating a lead hyper-precipitating strain of Caulobacter crescentus, the method comprising exposing bacteria of a first strain of Caulobacter crescentus to a mutagen, thereafter selecting exposed bacteria which exhibit greater cysteine synthase activity than do bacteria of the first strain, and culturing the selected bacteria to induce their proliferation, whereby the proliferated bacteria are the lead hyper-precipitating strain.

14. The method of claim 13, wherein the bacteria which exhibit greater cysteine synthase activity are selected by plating the proliferating bacteria on growth medium containing lead and selecting only cells that produce a visible brown color.

15. The method of claim 13, wherein bacteria originally auxotrophic for cysteine synthesis which exhibit greater cysteine synthase activity subsequent to selection on growth medium lacking cysteine.

16. The method of claim 13, wherein the cysteine synthesis is assessed by an enzyme assay.

17. A method of generating a lead hyper-precipitating strain of Caulobacter crescentus, the method comprising exposing bacteria of a first strain of Caulobacter crescentus to a mutagen culturing exposed bacteria in the presence of soluble lead, and thereafter selecting cultured bacteria which exhibit greater lead precipitation than do bacteria of the first strain, whereby the selected bacteria are the lead hyper-precipitating strain.

18. The method of claim 17, wherein lead precipitation is assessed by observing precipitation of lead hexaphosphate (LHP).

19. The method of claim 17, wherein lead precipitation is assessed by observing formation of darker bacterial colonies on a solid growth medium including soluble lead.

20. A method of reducing the amount of soluble lead in an aqueous fluid, the method comprising contacting the fluid with the lead hyper-precipitating strain of Caulobacter crescentus of claim 7 and thereafter separating the liquid and the strain.

21. The method of claim 20, further comprising recovering lead from the strain by cell lysis and separation of the precipitated lead from the insoluble fraction of the lysed cells.

22. A method of synthesizing lead hexaphosphate, comprising contacting a lead-containing fluid with the lead hyper-precipitating strain of Caulobacter crescentus of claim 7 and isolating the precipitated lead hexaphosphate.

23. A method of generating a metal hyper-precipitating behavior of Caulobacter crescentus, the method comprising exposing bacteria of a first strain of Caulobacter crescentus to a cysteine rich medium, wherein the medium contains a cysteine concentration ranging from about 0.01 to 1000 μg/ml, and thereafter selecting exposed bacteria which exhibit greater cysteine synthesis than do bacteria of the first strain, and culturing the selected bacteria to induce their proliferation, whereby the proliferated bacteria are the metal hyper-precipitating strain.

24. The method of claim 23, wherein the cysteine synthesis is assessed by an enzyme assay.

25. The method of claim 24, wherein the metal is lead, mercury, zinc, copper, cadmium, manganese, chromium, cobalt, nickel, silver or arsenic.

26. The method of claim 23, wherein the cysteine concentration ranges from 0.05 to 750 μg/ml,

27. The method of claim 26, wherein the cysteine concentration ranges from 0.10 to 500 μg/ml.

28. The method of claim 27, wherein the cysteine concentration is selected from the group consisting of 50, 100, 125, 150, 200, and 250 μg/ml.

29. A mutant CC1117 cysteine synthase protein having one or more amino acid sequences selected from the group consisting of SEQ ID NO. 7, SEQ ID No. 8, SEQ ID No. 9, and SEQ ID NO. 10.

30. A mutant CC1117 cysteine synthase having one or more nucleotide sequences selected from the group consisting of SEQ ID NO: 17, SEQ ID NO. 18, SEQ ID NO. 19, and SEQ ID NO. 20.
